(19) United States
(12) Patent Application Publication
(10) Pub. No.: US 2025/0043260 A1
(43) Pub. Date: Feb. 6, 2025

(54) Engineered High Activity Omni-79 Nuclease Variants

(71) Applicant: EMENDOBIO INC., Wilmington, DE (US)

(72) Inventors: Lior IZHAR, Tel Aviv (IL); Rafi EMMANUEL, Ramla (IL); Liat ROCKAH, Rishon LeZion (IL); Milit MAROM DAVID, Tel Aviv (IL)

(21) Appl. No.: 18/715,759

(22) PCT Filed: Nov. 30, 2022
(86) PCT No.: PCT/US2022/080627

§ 371 Date: Jun. 3, 2024

Related U.S. Application Data
(60) Provisional application No. 63/284,858, filed on Dec. 1, 2021.

(51) Int. Cl. C12N 9/22; C12N 15/11

(52) U.S. Cl. CPC C12N 9/22; C12N 15/111; C07K 2319/09; C12N 2310/20

(57) ABSTRACT

The present invention is directed to, inter alia, composition and methods for genome editing. Specifically, a non-naturally occurring OMNI-79 nuclease variant having a wild-type OMNI-79 protein sequence (SEQ ID NO: 1) comprising an amino acid substitution in at least one of the following positions: I14. S1005, and E1050.

This application claims the benefit of U.S. Provisional Application No. 63/284,858, filed Dec. 1, 2021, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “221130_91808-A-PCT_Sequence_Listing_AWG.xml”, which is 133 kilobytes in size, and which was created on Nov. 14, 2022 in the IBM-PC machine format, having an operating system compatibility with MS-Windows, which is contained in the XML file filed Nov. 30, 2022 as part of this application.

### BACKGROUND OF INVENTION

Targeted genome modification is a powerful tool that can be used to reverse the effect of pathogenic genetic variations and therefore has the potential to provide new therapies for human genetic diseases. Current genome engineering tools, including engineered zinc finger nucleases (ZFNs), transcription activator-like effector nucleases (TALENs), and most recently, RNA-guided DNA endonucleases such as CRISPR/Cas, produce sequence-specific DNA breaks in a genome. The modification of the genomic sequence occurs at the next step and is the product of the activity of a cellular DNA repair mechanism triggered in response to the newly formed DNA break. These mechanisms may include, for example: (1) classical non-homologous end-joining (NHEJ) in which the two ends of the break are ligated together in a fast but also inaccurate manner (i.e. frequently resulting in mutation of the DNA at the cleavage site in the form of small insertion or deletions) or (2) homology-directed repair (HDR) in which an intact homologous DNA donor is used to replace the DNA surrounding the cleavage site in an accurate manner. Minimal off-target activity of the initial DNA damage inducer is required for efficient and safe genome editing.

### SUMMARY OF THE INVENTION

Disclosed herein are engineered Clustered Regularly Interspaced Short Palindromic Repeats (CRISPRs)/CRISPR-associated OMNI-79 nucleases with improved activity and their use in genomic engineering, epigenomic engineering, genome targeting, genome editing, and in vitro diagnostics.

In some embodiments, there is provided a variant of an OMNI-79 nuclease with increased activity as compared to the wild-type OMNI-79 nuclease, as well as methods of using the improved variants. Advantageously, when the engineered variant OMNI-79 nucleases are active in a CRISPR endonuclease system, and the CRISPR endonuclease system displays increased on-target editing activity relative to a wild-type CRISPR endonuclease system in which a wild-type OMNI-79 nuclease is active. For example, an engineered variant OMNI-79 nuclease may display improved nuclease activity at a target region which contains a heterozygous SNP present in only the targeted allele and not present in the non-targeted allele.

According to some embodiments of the present invention, there is provided a variant of OMNI-79 nuclease protein comprising a sequence that is at least 80% identical to the amino acid sequence of wild-type OMNI-79 nuclease protein (SEQ ID NO: 1).

According to some embodiments of the present invention, there is provided a non-naturally occurring OMNI-79 nuclease variant having a wild-type OMNI-79 protein sequence (SEQ ID NO: 1) comprising an amino acid substitution in at least one of the following positions: I14, S1005, and E1050.

According to some embodiments of the present invention, there is provided a CRISPR system comprising any one of the OMNI-79 nuclease variants disclosed herein complexed with a guide RNA molecule that targets a DNA target site, wherein the CRISPR system displays increased on-target editing activity relative to a wild-type CRISPR system comprising a wild-type OMNI-79 nuclease protein and the guide RNA molecule.

In some embodiments, the OMNI-79 variant nuclease exhibits increased activity at a target site when complexed with a guide RNA targeting the OMNI-79 variant to the target site compared to a wild-type OMNI-79 nuclease (SEQ ID NO: 1).

According to some embodiments of the present invention, there is provided a method for gene editing having increased on-target editing activity, comprising contacting a DNA target site with an active CRISPR system comprising any one of the OMNI-79 nuclease variant proteins described herein.

According to some embodiments, there is provided a method for gene editing increased on-target editing activity, comprising:
contacting a target site with an active CRISPR system comprising a variant OMNI-79 nuclease protein of any one of the variants described herein, wherein the active CRISPR system displays increased on-target editing activity relative to a wild-type CRISPR system having a wild-type OMNI-79 nuclease protein.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Wild-type OMNI-79 nuclease and OMNI-79 Variant 5570 editing activity on various hLDLR gene targets. Editing activity was determined by next-generation sequencing (NGS) analysis. The average and standard deviation of three replicates is displayed.

FIGS. 2A-2C: Editing activity of OMNI-79 single mutants. Single point mutation variants were tested via transfection in HeLa cells targeting three genomic sites: (FIG. 2A) hSERP_g12R, (FIG. 2B) hLDLR_g46 and (FIG. 2C) hLDLR_g76. Editing activity was determined by NGS analysis. The average and standard deviation of three replicates is displayed.

FIG. 3: The effect of other substitutions at position 1005 on editing activity. The editing activity was tested via transfection in HeLa cells targeting hLDLR_g76. Editing activity was determined by NGS analysis. The average and standard deviation of three replicates is displayed.

FIG. 4: NGS analysis of editing by OMNI-79 V5570 RNP complexes. HepG2 (human hepatic carcinoma) cells were electroporated with preassembled purified OMNI-79 V5570 protein (105 μmol) and sgRNA (124 μmol) complexes using a Lonza Nucleofector X unit according to manufacturer's instructions. Following recovery, cells were plated in 12-well tissue culture plates and kept in a 37° C., 5% CO₂ incubator. 72 hours after electroporation, cells were dissociated with trypsin and genomic extract was prepared with QuickExtract solution according to manufacturer's instructions. InDels percentage analysis was performed by NGS.

FIG. 5: NGS analysis of editing by OMNI-79 V5570 mRNA in HepG2 cells. HepG2 cells were electroporated with 1 μg of OMNI-79 V5570-encoding mRNA (Trilink), and 124 pmol sgRNA (Agilent) using a Lonza Nucleofector X unit according to manufacturer's instructions. Following recovery, cells were plated in 12-well tissue culture plates and kept in 37° C., 5% CO₂ incubator. 72 hours after electroporation, cells were dissociated with trypsin and genomic extract was prepared with QuickExtract solution according to manufacturer's instructions. InDels percentage analysis was performed by NGS.

FIG. 6: NGS analysis of editing by OMNI-79 V5570 and gRNA expressed by AAV infection. Hepa1-6 (mouse hepatoma) cells and HepG2 cells were infected with Adeno-associated viruses-DJ serotype (AAV-DJ) harboring OMNI-79 V5570 and corresponding sgRNA sequences at a multiplicity of infection (MOI) of 1×10⁵-3×10⁵. Cells were plated in 12-well tissue culture plates and kept in a 37° C., 5% CO₂ incubator for 16 hours, then washed from remaining viruses and incubated for another 48 hours with fresh medium. Cells were dissociated with trypsin and genomic extract was prepared with QuickExtract solution according to manufacturer's instructions. InDels percentage analysis was performed by NGS.

FIG. 7: NGS analysis of editing by OMNI-79 V5570 mRNA in Hela cells. The editing activity was tested via transfection of OMNI-79 V5570-encoding mRNA (in-house IVT) in Hela cells targeting hSERP_g12 and CXCR4_s25 sites. Editing activity was determined by NGS analysis. The average and standard deviation of three replicates is displayed.

### DETAILED DESCRIPTION

The present disclosure provides an engineered OMNI-79 nuclease exhibiting increased activity at a target site compared to the wild-type OMNI-79 nuclease (SEQ ID NO: 1). The wild-type OMNI-79 nuclease is disclosed in PCT International Application No. PCT/US2021/035928, incorporated herein by reference. When the engineered OMNI-79 nuclease variant is active in a CRISPR endonuclease system, the CRISPR endonuclease system displays increased on-target editing activity relative to a CRISPR endonuclease system comprising the wild-type OMNI-79 nuclease. In some embodiments, the engineered OMNI-79 nuclease is an OMNI-79 nuclease variant comprising at least one amino acid substitution relative to the wild-type OMNI-79 nuclease. In some embodiments, the engineered OMNI-79 nuclease comprises multiple amino acid substitutions compared to wild-type OMNI-79 nuclease.

In some embodiments, and OMNI-79 nuclease variant is at least 80%, e.g., at least 85%, 86%, 87%, 88%, 89%, 90%, 91%, 92%, 93%, 94%, 95%, 96%, 97%, 98% or 99% identical to the amino acid sequence of SEQ ID NO: 1. As a non-limiting example, an OMNI-79 nuclease variant may have amino acid sequence differences at up to 1%, 2%, 3%, 4%, 5%, 6%, 7%, 8%, 9%, 10%, 11%, 12%, 13%, 14%, 15%, or 20% of its residues relative to SEQ ID NO:1. Such sequence differences may be revealed by a sequence alignment. An OMNI-79 variant nuclease may be generated by replacing at least one amino acid residue of an OMNI-79 wild-type nuclease with another amino acid residue e.g. with a conservative or non-conservative amino acid substitution, and/or by inserting or deleting an amino acid residue of the OMNI-79 wild-type nuclease. Any such mutations, including but not limited to substitutions, insertions, or deletions, in addition to any other mutations described herein, or with mutations in addition to the mutations described herein, may be used to generate an OMNI-79 variant nuclease from an OMNI-79 wild-type nuclease. In some embodiments, the OMNI-79 variant nuclease retains a desired activity of the parent wild-type OMNI-79 nuclease, e.g., the ability to interact with a guide RNA and target DNA and/or the activity of the nuclease (e.g. ability to cause a double-strand DNA break, a single-strand DNA break, or lack of any nuclease or nickase activity). In some embodiments, the variant retains the desired activity of the parent, e.g. nuclease activity, at a level greater than or equal to the level of activity of the parent. In some embodiments, the variant retains the desired activity of the parent at a level of at least 100%, 95%, 90%, 80%, 70%, 60%, 50%, 40%, or 30% the level of activity of the parent. In some embodiments, the OMNI-79 variant nuclease displays increased on-target effects relative to OMNI-79 wild-type nuclease. In some embodiments, there is provided a variant of an OMNI-79 nickase with increased activity as compared to the wild-type OMNI-79 nickase. In some embodiments, there is provided a variant of an OMNI-79 dead nuclease with increased activity as compared to the wild-type OMNI-79 dead nuclease. In some embodiments, the OMNI-79 nuclease variant is a nickase having an inactivated RuvC or HNH domain and further comprises an amino acid substitution in at least one of the following positions: S1005, I14 and E1050. In some embodiments, the OMNI-79 nuclease variant is a dead nuclease having an inactivated RuvC and HNH domains and further comprises an amino acid substitution in at least one of the following positions: S1005, I14 and E1050.

In some embodiments, there is provided a variant of OMNI-79 nuclease protein comprising a sequence that is at least 80% identical to the amino acid sequence of wild-type OMNI-79 (SEQ ID NO: 1) and having at least one amino acid substitution. In some embodiments, the amino acid substitution comprises an amino acid residue replacement to a positive, negative, uncharged, hydrophilic, hydrophobic, polar, or non-polar amino acid. In some embodiments, the amino acid substitution is selected from replacement of an amino acid to any one of a different amino acid selected from the group consisting of R, K, H, D, E, S, T, N, Q, C, U, G, P, A, I, L, M, F, W, Y and V.

In some embodiments, a variant OMNI-79 nuclease protein contains an amino acid substitution in at least one of the following positions in the wild-type OMNI-79 protein sequence (SEQ ID NO:1): I14, S1005, and E1050. Each possibility represents a separate embodiment of the present disclosure. In some embodiments, there is provided a variant of OMNI-79 nuclease protein comprising a sequence that is at least 80% identical to the amino acid sequence of the wild-type OMNI-79 nuclease (SEQ ID NO: 1) and having at least one amino acid substitution in at least one of the following positions in the wild-type OMNI-79 protein sequence: I14, S1005, and E1050. In some embodiments, the variant OMNI-79 nuclease protein comprises at least one of the following amino acid substitutions in the following positions in the wild-type OMNI-79 protein sequence: I14L, S1005R, S1005K, and E1050K. Each possibility represents a separate embodiment of the present disclosure. In some embodiments, the substitution corresponds to those listed in Table 3.

In some embodiments, the variant OMNI-79 nuclease protein comprises at least one amino acid substitution in the following positions in the wild-type OMNI-79 protein sequence: I14 and S1005. In some embodiments, the variant OMNI-79 nuclease protein comprises amino acid substitutions in the following positions in the wild-type OMNI-79 protein sequence: I14 and S1005. In some embodiments, the variant OMNI-79 nuclease protein comprises the following amino acid substitutions from the wild-type OMNI-79 protein sequence: I14L and S1005R.

In some embodiments, the variant OMNI-79 nuclease protein comprises at least one amino acid substitution in the following positions in the wild-type OMNI-79 protein sequence: S1005 and E1050. In some embodiments, the variant OMNI-79 nuclease protein comprises amino acid substitutions in the following positions in the wild-type OMNI-79 protein sequence: S1005 and E1050. In some embodiments, the variant OMNI-79 nuclease protein comprises the following amino acid substitutions from the wild-type OMNI-79 protein sequence: S1005K and E1050K.

In some embodiments, the OMNI-79 variant nuclease further comprises one or more of a nuclear localization sequence (NLS), cell penetrating peptide sequence, and/or affinity tag. In an embodiment, the OMNI-79 variant nuclease comprises one or more nuclear localization sequences of sufficient strength to drive accumulation of a CRISPR complex comprising the CRISPR nuclease in a detectable amount in the nucleus of a eukaryotic cell.

In some embodiments, the OMNI-79 variant nuclease comprises amino acid substitutions selected from amino acid substitutions corresponding to the substitutions displayed relative to wild-type OMNI-79 in Table 3.

According to some embodiments, there is provided an isolated OMNI-79 variant nuclease protein comprising one or more substitutions or mutations relative to the wild-type OMNI-79 nuclease sequence, wherein the isolated variant OMNI-79 variant nuclease is active in a CRISPR system, wherein the CRISPR system displays increased on-target editing activity relative to a wild-type CRISPR system.

According to some embodiments, additional mutations to the OMNI-79 variant nuclease described herein may be implemented. Examples include, but are not limited to, mutations which alter the PAM recognition sequence, alter the nuclease activity of the enzyme, and truncations or removal of portions of the nuclease. According to some embodiments, the variant OMNI-79 variant nuclease may be encoded by any nucleic acid sequence which produces the desired amino acid sequence of the variant. For example, the nucleic acid sequence may be codon-optimized for a cell, such as a bacterial cell, plant cell, or mammalian cell.

In embodiments of the present invention, a CRISPR nuclease and a targeting molecule form a CRISPR complex that binds to a target DNA sequence to effect cleavage of the target DNA sequence. A CRISPR nuclease may form a CRISPR complex comprising the CRISPR nuclease and a single-guide RNA (sgRNA) molecule. Alternatively, a CRISPR nucleases may form a CRISPR complex comprising the CRISPR nuclease, a crRNA molecule, and a tracrRNA molecule.

According to some embodiments of the present invention, there is provided a method of gene editing having increased on-target editing activity, comprising: contacting a target site with an active CRISPR endonuclease system having a variant OMNI-79 protein complexed with a suitable guide RNA or guide RNA complex, wherein the active CRISPR endonuclease system displays increased on-target editing activity relative to a wild-type OMNI-79 CRISPR system.

According to some embodiments, there is provided a non-naturally occurring OMNI-79 nuclease variant having a wild-type OMNI-79 protein sequence (SEQ ID NO: 1) comprising an amino acid substitution in at least one of the following positions: I14, S1005, and E1050.

In some embodiments, the amino acid substitution at S1005 and/or E1050 are to an amino acid having a positively charged R-group.

In some embodiments, the amino acid having a positively charged R-group is lysine or arginine.

In some embodiments, the amino acid substitution is any one of the following substitutions: I14L, S1005R, S1005K, and E1050K.

In some embodiments, the OMNI-79 nuclease variant comprises an amino acid substitution at each of positions I14 and S1005.

In some embodiments, the amino acid substitutions are I14L and S1005R.

In some embodiments, the OMNI-79 nuclease variant comprises an amino acid substitution at each of positions S1005 and E1050.

In some embodiments, the amino acid substitutions are S1005K and E1050K.

In some embodiments, the OMNI-79 nuclease variant has an amino acid sequence of any one of SEQ ID NO: 2, SEQ ID NO: 3, or SEQ ID NOs: 12-25.

In some embodiments, the amino acid substitution is at I14 and is any one of the following substitutions: I14L, I14V, I14F, I14C, I14A, or I14T.

In some embodiments, the amino acid substitution is I14L.

In some embodiments, the amino acid substitution is at I14 and the amino acid has an aromatic or hydrophobic R-group.

In some embodiments, the amino acid substitution is at S1005 and is any one of the following substitutions: S1005R, S1005K, S1005Q, S1005I, S1005M, S1005V, S1005T, S1005N, S1005F, S1005A, S1005G, or S1005E.

In some embodiments, the amino acid substitution at S1005 is to an amino acid having a positively charged R-group.

In some embodiments, the amino acid substitution at S1005 is to an amino acid having a polar R-group.

In some embodiments, the amino acid substitution is S1005R.

In some embodiments, the amino acid substitution is S1005K.

In some embodiments, the amino acid substitution is S1005T

In some embodiments, the amino acid substitution is S1005N.

In some embodiments, the amino acid substitution is S1005Q.

In some embodiments, the amino acid substitution is at E1050 and is any one of the following substitutions: E1050K, E1050R, E1050P, E1050A, E1050I, E1050L, E1050V, E1050G, or E1050T.

In some embodiments, the amino acid substitution is E1050K.

In some embodiments, the amino acid substitution at E1050 is to an amino acid having a positively charged R-group.

In some embodiments, the OMNI-79 nuclease variant has at least 80% sequence identity to the wild-type OMNI-79 protein sequence (SEQ ID NO:1). For example, in some embodiments the OMNI-79 nuclease variant may have at least 80% sequence identity to the wild-type OMNI-79 protein sequence (SEQ ID NO:1) and any one of the amino acid substitutions provided herein.

In some embodiments, the OMNI-79 nuclease variant may comprise any one of the amino acid substitutions provided herein relative to SEQ ID NO: 1, with the remaining amino acid sequence having at least 80% sequence identity to the wild-type OMNI-79 protein sequence (SEQ ID NO:1).

In some embodiments, the OMNI-79 nuclease variant further comprises a nuclear localization sequence (NLS).

In some embodiments, the OMNI-79 nuclease variant exhibits increased activity toward a DNA target site when complexed with a guide RNA molecule that targets the variant to the said DNA target site relative to a wild-type OMNI-79 nuclease complexed with the guide RNA molecule.

According to some embodiments of the present invention, there is provided a CRISPR system comprising any one of the OMNI-79 nuclease variants described herein complexed with a guide RNA molecule that targets a DNA target site, wherein the CRISPR system displays increased on-target editing activity relative to a wild-type CRISPR system comprising a wild-type OMNI-79 nuclease protein and the guide RNA molecule.

According to some embodiments of the present invention, there is provided a method for gene editing having increased on-target editing activity, comprising contacting a DNA target site with an active CRISPR system comprising any one of the OMNI-79 nuclease variant proteins described herein.

In some embodiments, the gene editing occurs in a eukaryotic cell or prokaryotic cell.

In some embodiments, the eukaryotic cell is a plant cell or mammalian cell.

In some embodiments, the mammalian cell is a human cell.

In some embodiments, the DNA target site is located within or in proximity to a pathogenic allele of a gene.

In some embodiments, the DNA target is repaired with an exogenous donor molecule.

In some embodiments, the on-target editing activity is increased by at least 10%, 20%, 30%, 40%, 50%, 60%, 70%, 80%, 90%, 2-fold, 10-fold, 102-fold, 103-fold, 104-fold, 10⁵-fold, or 106-fold.

According to some embodiments of the present invention, there is provided a modified cell obtained by the methods described herein.

In some embodiments, the cell is capable of engraftment.

In some embodiments, the cell is capable of giving rise to progeny cells after engraftment.

In some embodiments, the cell is capable of giving rise to progeny cells after an autologous engraftment.

In some embodiments, the cell is capable of giving rise to progeny cells for at least 12 months or at least 24 months after engraftment.

In some embodiments, the cell is selected from the group consisting of a hematopoietic stem cell, a progenitor cell, a CD34+ hematopoietic stem cell, a bone marrow cell, and a peripheral mononucleated cell.

According to some embodiments of the present invention, there is provided a composition comprising any one of the modified cells described herein and a pharmaceutically acceptable carrier. According to some embodiments of the present invention, there is provided an in vitro or ex vivo method of preparing the composition, comprising mixing the cells with the pharmaceutically acceptable carrier.

According to some embodiments of the present invention, there is provided a polynucleotide molecule encoding any one of the OMNI-79 nuclease variant proteins described herein.

### Delivery

The OMNI-79 variant compositions described herein may be delivered as a protein, DNA molecules, RNA molecules, Ribonucleoproteins (RNP), nucleic acid vectors, or any combination thereof. In some embodiments, the RNA molecule comprises a chemical modification. Non-limiting examples of suitable chemical modifications include 2′-O-methyl (M), 2′-O-methyl, 3′phosphorothioate (MS) or 2′-O-methyl, 3′thioPACE (MSP), pseudouridine, and 1-methyl pseudo-uridine. Each possibility represents a separate embodiment of the present invention.

The OMNI-79 variants and/or polynucleotides encoding same described herein, and/or additional molecules, such as a single-guide RNA molecule, crRNA molecule, tracrRNA molecules or a nucleotide molecule that encodes any one of them, may be delivered to a target cell by any suitable means. The target cell may be any type of cell e.g., eukaryotic or prokaryotic, in any environment e.g., isolated or not, maintained in culture, in vitro, ex vivo, in vivo or in planta. A target site in a target cell may be within the nucleus of the cell.

The compositions described herein may be introduced into a cell as part of a vector molecule having additional sequences such as, for example, replication origins, promoters and genes encoding antibiotic resistance. Moreover, compositions may introduced into a cell as naked nucleic acids or proteins, as nucleic acids or proteins complexed with or packaged within an agent such as a liposome, exosome, or poloxamer, or can be delivered by recombinant viruses (e.g., adenovirus, AAV, herpesvirus, retrovirus, lentivirus and integrase defective lentivirus (IDLV)) or virus-like particles. As non-limiting examples, the composition may be packaged into an adeno-associated virus (AAV), or into a lentivirus, such as a non-integrating lentivirus or a lentivirus lacking reverse transcription capability. Additional non-limiting examples include packaging the composition into liposomes, extracellular vesicles, or exosomes, which may be pseudotyped with vesicular stomatitis glycoprotein (VSVG) or conjugated to a cell-penetrating peptide, an antibody, a targeting moiety, or any combination thereof.

In some embodiments, the composition to be delivered includes mRNA of the nuclease and RNA of the guide. In some embodiments, the composition to be delivered includes mRNA of the nuclease, RNA of the guide and a donor template. In some embodiments, the composition to be delivered includes the CRISPR nuclease and guide RNA. In some embodiments, the composition to be delivered includes the CRISPR nuclease, guide RNA and a donor template for gene editing via, for example, homology directed repair. Optionally the lentivirus includes mRNA of the nuclease and a guide RNA molecule, e.g. a single-guide RNA molecule or crRNA molecule, which is used to target the nuclease to a target site. In some embodiments, the composition delivered to a cell includes mRNA of the nuclease, a guide RNA molecule and a donor template molecule. Optionally, the lentivirus includes the nuclease protein variant and a guide RNA molecule. Optionally, the composition delivered to a cell includes the nuclease protein variant, a guide RNA molecule and/or donor template for homology directed repair. Optionally, the composition delivered to a cell includes mRNA of the nuclease variant, a DNA-targeting crRNA molecule, and a tracrRNA molecule, the composition delivered to a cell includes mRNA of the nuclease variant, DNA-targeting crRNA molecule, and a tracrRNA molecule, and a donor template molecule, the composition delivered to a cell includes the nuclease protein variant, DNA-targeting crRNA molecule, and a tracrRNA molecule. Optionally, the composition delivered to a cell includes the nuclease protein variant, DNA-targeting crRNA molecule, and a tracrRNA molecule, and DNA donor template molecule for homology directed repair.

Any suitable viral vector system may be used to deliver such compositions. Conventional viral and non-viral based gene transfer methods can be used to introduce nucleic acids and/or OMNI-79 nuclease variant protein in cells (e.g., mammalian cells, plant cells, etc.) and target tissues. Such methods can also be used to administer nucleic acids encoding and/or OMNI-79 nuclease variant protein to cells in vitro. In certain embodiments, nucleic acids and/or an OMNI-79 nuclease variant protein are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology Doerfler and Bohm (eds.) (1995); and Yu et al., Gene Therapy 1:13-26 (1994).

Methods of non-viral delivery of nucleic acids and/or proteins include electroporation, lipofection, microinjection, biolistics, particle gun acceleration, virosomes, virus-like particles, exosomes, liposomes, immunoliposomes, polycation or lipid: nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus. See, e.g., Chung et al. Trends Plant Sci. (2006). Sonoporation using, e.g., the Sonitron 2000 system (Rich-Mar) can also be used for delivery of nucleic acids. Cationic-lipid mediated delivery of proteins and/or nucleic acids is also contemplated as an in vivo or in vitro delivery method. See Zuris et al., Nat. Biotechnol. (2015), Coelho et al., N. Engl. J. Med. (2013); Judge et al., Mol. Ther. (2006); and Basha et al., Mol. Ther. (2011).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386, 4,946,787; and 4,897,355) and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiamid et al (2009) Nature Biotechnology 27 (7) p. 643).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues. An OMNI-79 variant or a nucleic acid expressing the variant, as well as any associated nucleic acids, may be delivered by a non-integrating lentivirus. Optionally, RNA delivery with lentivirus is utilized. Optionally, the lentivirus includes mRNA of the nuclease and a guide RNA molecule, e.g. a single-guide RNA molecule or crRNA molecule, which is used to target the nuclease to a target site. Optionally the lentivirus includes mRNA of the nuclease, guide RNA molecule and a donor template molecule. Optionally, the lentivirus includes the nuclease protein variant and a guide RNA molecule. Optionally, the lentivirus includes the nuclease protein variant, a guide RNA molecule and/or donor template molecule for homology directed repair. Optionally, the lentivirus includes mRNA of the nuclease variant, a DNA-targeting crRNA molecule, and a tracrRNA molecule. Optionally the lentivirus includes mRNA of the nuclease variant, DNA-targeting crRNA molecule, and a tracrRNA molecule, and a donor template molecule. Optionally, the lentivirus includes the nuclease protein variant, DNA-targeting crRNA molecule, and a tracrRNA molecule. Optionally, the lentivirus includes the nuclease protein variant, DNA-targeting crRNA molecule, and a tracrRNA molecule, and DNA donor template molecule for homology directed repair.

As mentioned above, the compositions described herein may be delivered to a target cell using a non-integrating lentiviral particle method, e.g. a LentiFlash® system. Such a method may be used to deliver mRNA or other types of RNAs into the target cell, such that delivery of the RNAs to the target cell results in assembly of the compositions described herein inside of the target cell. See also PCT International Publication Nos. WO2013/014537, WO2014/016690, WO2016185125, WO2017194902, and WO2017194903.

The tropism of a retrovirus can be altered by incorporating foreign envelope proteins, expanding the potential target population of target cells. Lentiviral vectors are retroviral vectors capable of transducing or infecting non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher Panganiban, J. Virol. (1992); Johann et al., J. Virol. (1992); Sommerfelt et al., Virol. (1990); Wilson et al., J. Virol. (1989); Miller et al., J. Virol. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood (1995); Kohn et al., Nat. Med. (1995); Malech et al., PNAS (1997)). PA317/pLASN was the first therapeutic vector used in a gene therapy trial. (Blaese et al., Science (1995)). Transduction efficiencies of 50% or greater have been observed for MFG-S packaged vectors. (Ellem et al., Immunol Immunother. (1997); Dranoff et al., Hum. Gene Ther. (1997).

Packaging cells are used to form virus particles that are capable of infecting a host cell. Such cells include 293 cells, which package adenovirus, AAV, and .psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with an RNA composition, and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

Suitable cells include but not limited to eukaryotic and prokaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOKISV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells, any plant cell (differentiated or undifferentiated) as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with nuclease systems (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma. and TNF-alpha are known (as a non-limiting example see, Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (as a non-limiting example see Inaba et al., J. Exp. Med. 176:1693-1702 (1992)). Stem cells that have been modified may also be used in some embodiments.

Notably, any one of the OMNI-79 variant described herein may be suitable for genome editing in post-mitotic cells or any cell which is not actively dividing, e.g., arrested cells. Examples of post-mitotic cells which may be edited using an OMNI-79 variant of the present invention include, but are not limited to, myocyte, a cardiomyocyte, a hepatocyte, an osteocyte and a neuron.

Vectors (e.g., retroviruses, liposomes, etc.) containing therapeutic RNA compositions can also be administered directly to an organism for transduction of cells in vivo. Alternatively, naked RNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of transgenes into immune cells (e.g., T-cells) include non-integrating lentivirus vectors. See, for example, U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

### DNA Repair by Homologous Recombination

In some embodiments of the present invention, a variant OMNI-79 nuclease is utilized to affect a DNA break at a target site to induce cellular repair mechanisms, for example, but not limited to, non-homologous end-joining (NHEJ) or homology-directed repair (HDR).

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length (or any integer value there between or there above), preferably between about 100 and 1,000 nucleotides in length (or any integer there between), more preferably between about 200 and 500 nucleotides in length. A nucleic acid template may be a single stranded nucleic acid, a double stranded nucleic acid. In some embodiment, the nucleic acid template comprises a nucleotide sequence, e.g., of one or more nucleotides, that corresponds to wild type sequence of the target nucleic acid, e.g., of the target position. In some embodiment, the nucleic acid template comprises a ribonucleotide sequence, e.g., of one or more ribonucleotides, that corresponds to wild type sequence of the target nucleic acid, e.g., of the target position. In some embodiment, the nucleic acid template comprises modified ribonucleotides.

Insertion of an exogenous sequence (also called a “donor sequence,” donor template” or “donor”), for example, for correction of a mutant gene or for increased expression of a wild-type gene can also be carried out. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. See, e.g., U.S. Patent Publication Nos. 2010/0047805; 2011/0281361; 2011/0207221; and 2019/0330620. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang and Wilson, Proc. Natl. Acad. Sci. USA (1987); Nehls et al., Science (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Accordingly, embodiments of the present invention using a donor template for repair may use a DNA or RNA, single-stranded and/or double-stranded donor template that can be introduced into a cell in linear or circular form. In embodiments of the present invention a gene-editing composition comprises: (1) an RNA molecule comprising a guide sequence to affect a double strand break in a gene prior to repair and (2) a donor RNA template for repair, and the RNA molecule comprising the guide sequence is a first RNA molecule and the donor RNA template is a second RNA molecule. In some embodiments, the guide RNA molecule and template RNA molecule are connected as part of a single molecule.

A donor sequence may also be an oligonucleotide and be used for gene correction or targeted alteration of an endogenous sequence. The oligonucleotide may be introduced to the cell on a vector, may be electroporated into the cell, or may be introduced via other methods known in the art. The oligonucleotide can be used to ‘correct’ a mutated sequence in an endogenous gene (e.g., the sickle mutation in beta globin), or may be used to insert sequences with a desired purpose into an endogenous locus.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with or packaged within an agent such as a liposome, exosome, or poloxamer, or can be delivered by recombinant viruses (e.g., adenovirus, AAV, herpesvirus, retrovirus, lentivirus and integrase defective lentivirus (IDLV)) or virus-like particles. Non-viral vectors, such as transposon-based systems, e.g. recombinant Sleeping Beauty transposon systems or recombinant PiggyBac transposon systems, may also be utilized for transposition of a polynucleotide sequence in a target cell.

The donor is generally inserted so that its expression is driven by the endogenous promoter at the integration site, namely the promoter that drives expression of the endogenous gene into which the donor is inserted. However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an endogenous locus such that some (N-terminal and/or C-terminal to the transgene) or none of the endogenous sequences are expressed, for example as a fusion with the transgene. In other embodiments, the transgene (e.g., with or without additional coding sequences such as for the endogenous gene) is integrated into any endogenous locus, for example a safe-harbor locus, for example a CCR5 gene, a CXCR4 gene, a PPPIR12c (also known as AAVS1) gene, an albumin gene or a Rosa gene. See, e.g., U.S. Pat. Nos. 7,951,925 and 8,110,379; U.S. Publication Nos. 2008/0159996; 20100/0218264; 2010/0291048; 2012/0017290; 2011/0265198; 2013/0137104; 2013/0122591; 2013/0177983 and 2013/0177960 and U.S. Provisional Application No. 61/823,689).

When endogenous sequences (endogenous or part of the transgene) are expressed with the transgene, the endogenous sequences may be full-length sequences (wild-type or mutant) or partial sequences. Preferably the endogenous sequences are functional. Non-limiting examples of the function of these full length or partial sequences include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

In certain embodiments, the donor molecule comprises a sequence selected from the group consisting of a gene encoding a protein (e.g., a coding sequence encoding a protein that is lacking in the cell or in the individual or an alternate version of a gene encoding a protein), a regulatory sequence and/or a sequence that encodes a structural nucleic acid such as a microRNA or siRNA.

### DNA-Targeting RNA Molecules

In embodiments of the present invention, a DNA-targeting RNA molecule comprises a guide sequence portion. The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. The entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the DNA molecule having the guide sequence portion is present contemporaneously with the CRISPR molecule the RNA molecule is capable of targeting the CRISPR nuclease to the specific target DNA sequence. Each possibility represents a separate embodiment. An RNA molecule can be custom designed to target any desired sequence. Accordingly, a molecule comprising a “guide sequence portion” is a type of targeting molecule. Throughout this application, the terms “guide molecule,” “RNA guide molecule,” “guide RNA molecule,” and “gRNA molecule” are synonymous with a molecule comprising a guide sequence portion, and the term “spacer” is synonymous with a “guide sequence portion.”

According to some aspects of the invention, the disclosed methods comprise a method of modifying a nucleotide sequence at a target site in a cell-free system or the genome of a cell comprising introducing into the cell the composition of any one of the embodiments described herein.

In some embodiments, the cell is a eukaryotic cell, preferably a mammalian cell or a plant cell. In some embodiments, genome modifying occurs within the nucleus of a cell.

According to some aspects of the invention, the disclosed methods comprise a use of any one of the compositions described herein for the treatment of a subject afflicted with a disease associated with a genomic mutation comprising modifying a nucleotide sequence at a target site in the genome of the subject.

According to some aspects of the invention, the disclosed methods comprise a method of treating subject having a mutation disorder comprising targeting any one of the compositions described herein to an allele associated with the mutation disorder.

In some embodiments, the mutation disorder is related to a disease or disorder selected from any of a neoplasia, age-related macular degeneration, schizophrenia, neurological, neurodegenerative, or movement disorder, Fragile X Syndrome, secretase-related disorders, prion-related disorders, ALS, addiction, autism, Alzheimer's Disease, neutropenia, inflammation-related disorders, Parkinson's Disease, blood and coagulation diseases and disorders, beta thalassemia, sickle cell anemia, cell dysregulation and oncology diseases and disorders, inflammation and immune-related diseases and disorders, metabolic, liver, kidney and protein diseases and disorders, muscular and skeletal diseases and disorders, dermatological diseases and disorders, neurological and neuronal diseases and disorders, and ocular diseases and disorders.

### Diseases and Therapies

Certain embodiments of the invention target a nuclease to a specific genetic locus associated with a disease or disorder as a form of gene editing, method of treatment, or therapy. For example, to induce editing or knockout of a gene, a novel nuclease disclosed herein may be specifically targeted to a pathogenic mutant allele of the gene using a custom designed guide RNA molecule. The guide RNA molecule is preferably designed by first considering the PAM requirement of the nuclease, which as shown herein is also dependent on the system in which the gene editing is being performed. For example, a guide RNA molecule designed to target an OMNI-79 nuclease to a target site is designed to contain a spacer sequence complementary to a DNA strand of a DNA double-stranded region that neighbors a OMNI-79 PAM sequence, e.g. “NGG.” The guide RNA molecule is further preferably designed to contain a spacer region (i.e. the region of the guide RNA molecule having complementarity to the target allele) of sufficient and preferably optimal length in order to increase specific activity of the nuclease and reduce off-target effects.

As a non-limiting example, the guide RNA molecule may be designed to target the nuclease to a specific region of a mutant allele, e.g. near the start codon, such that upon DNA damage caused by the nuclease a non-homologous end joining (NHEJ) pathway is induced and leads to silencing of the mutant allele by introduction of frameshift mutations. This approach to guide RNA molecule design is particularly useful for altering the effects of dominant negative mutations and thereby treating a subject. As a separate non-limiting example, the guide RNA molecule may be designed to target a specific pathogenic mutation of a mutated allele, such that upon DNA damage caused by the nuclease a homology directed repair (HDR) pathway is induced and leads to template mediated correction of the mutant allele. This approach to guide RNA molecule design is particularly useful for altering haploinsufficiency effects of a mutated allele and thereby treating a subject.

Non-limiting examples of specific genes which may be targeted for alteration to treat a disease or disorder are presented herein below. Specific disease-associated genes and mutations that induce a mutation disorder are described in the literature. Such mutations can be used to design a DNA-targeting RNA molecule to target a CRISPR composition to an allele of the disease associated gene, where the CRISPR composition causes DNA damage and induces a DNA repair pathway to alter the allele and thereby treat the mutation disorder.

Mutations in the ELANE gene are associated with neutropenia. Accordingly, without limitation, embodiments of the invention that target ELANE may be used in methods of treating subjects afflicted with neutropenia. Guide RNA molecules which target the ELANE gene and are useful for treating neutropenia are disclosed in PCT International Application No. PCT/US2020/059186, incorporated herein by reference.

CXCR4 is a co-receptor for the human immunodeficiency virus type 1 (HIV-1) infection. Accordingly, without limitation, embodiments of the invention that target CXCR4 may be used in methods of treating subjects afflicted with HIV-1 or conferring resistance to HIV-1 infection in a subject.

Programmed cell death protein 1 (PD-1) disruption enhances CAR-T cell mediated killing of tumor cells and PD-1 may be a target in other cancer therapies. Accordingly, without limitation, embodiments of the invention that target PD-1 may be used in methods of treating subjects afflicted with cancer. In an embodiment, the treatment is CAR-T cell therapy with T cells that have been modified according to the invention to be PD-1 deficient.

In addition, BCL11A is a gene that plays a role in the suppression of hemoglobin production. Globin production may be increased to treat diseases such as thalassemia or sickle cell anemia by inhibiting BCL11A. See for example, PCT International Publication No. WO 2017/077394A2; U.S. Publication No. US2011/0182867A1; Humbert et al. Sci. Transl. Med. (2019); and Canver et al. Nature (2015). Accordingly, without limitation, embodiments of the invention that target an enhancer of BCL11A may be used in methods of treating subjects afflicted with beta thalassemia or sickle cell anemia.

Embodiments of the invention may also be used for targeting any disease-associated gene, for studying, altering, or treating any of the diseases or disorders listed in Table A or Table B below. Indeed, any disease-associated with a genetic locus may be studied, altered, or treated by using the nucleases disclosed herein to target the appropriate disease-associated gene, for example, those listed in U.S. Publication No. 2018/0282762A1 and European Patent No. EP3079726B1.

TABLE A
Diseases, Disorders and their associated genes

| DISEASE/ | |
|---|---|
| DISORDERS | GENE(S) |
| Neoplasia | PTEN; ATM; ATR; EGFR; ERBB2; ERBB3; |
| | ERBB4; Notch1; Notch2; Notch3; Notch4; |
| | AKT; AKT2; AKT3; HIF; HIF1a; HIF3a; Met; |
| | HRG; Bcl2; PPAR alpha; PPAR gamma; WT1 |
| | (Wilms Tumor); FGF Receptor Family |
| | members (5 members: 1, 2, 3, 4, 5); CDKN2a; |
| | APC; RB (retinoblastoma); MEN1; VHL; |
| | BRCA1; BRCA2; AR (Androgen Receptor); |
| | TSG101; IGF; IGF Receptor; Igf1 (4 variants); |
| | gf2 (3 variants); Igf 1 Receptor; Igf 2 Receptor; |
| | Bax; Bcl2; caspases family (9 members: 1, 2, 3, |
| | 4, 6, 7, 8, 9, 12); Kras; Apc |
| Age-related Macular | Abcr; Ccl2; Cc2; cp (ceruloplasmin); Timp3; |
| Degeneration | cathepsinD; Vldlr; Ccr2 |
| Schizophrenia | Neuregulin1 (Nrg1); Erb4 (receptor for |
| | Neuregulin); Complexin1 (Cp1x1); Tph1 |
| | Tryptophan hydroxylase; Tph2 Tryptophan |
| | hydroxylase 2; Neurexin 1; GSK3; GSK3a; |
| | GSK3b |
| Neurological, Neuro | 5-HTT (S1c6a4); COMT; DRD (Drd1a); |
| degenerative, and | SLC6A3; DAOA; DTNBP1; Dao (Dao1) |
| Movement Disorders | |
| Trinucleotide Repeat | HTT (Huntington's Dx); SBMA/SMAX1/AR |
| Disorders | (Kennedy's Dx); FXN/X25 (Friedrich's Ataxia); |
| | ATX3 (Machado-Joseph's Dx); ATXN1 and |
| | ATXN2 (spinocerebellar ataxias); DMPK |
| | (myotonic dystrophy); Atrophin-1 and Atn1 |
| | (DRPLA Dx); CBP (Creb-BP-global instability); |
| | VLDLR (Alzheimer's); Atxn7; Atxn10 |
| Fragile X Syndrome | FMR2; FXR1; FXR2; mGLUR5 |
| Secretase Related | APH-1 (alpha and beta); Presenilin (Psen1); |
| Disorders | nicastrin (Ncstn); PEN-2 |
| Others | Nos1; Parp1; Nat1; Nat2 |
| Prion related disorders | Prp |
| ALS | SOD1; ALS2; STEX; FUS; TARDBP; VEGF |
| | (VEGF-a; VEGF-b; VEGF-c) |
| Addiction | Prkce (alcohol); Drd2; Drd4; ABAT (alcohol); |
| | GRIA2; Grm5; Grin1; Htr1b; Grin2a; Drd3; |
| | Pdyn; Gria1 (alcohol) |
| Autism | Mecp2; BZRAP1; MDGA2; Sema5A; Neurexin |
| | 1; Fragile X (FMR2 (AFF2); FXR1; FXR2; |
| | Mglur5) |
| Alzheimer's Disease | E1; CHIP; UCH; UBB; Tau; LRP; PICALM; |
| | Clusterin; PS1; SORL1; CR1; Vldlr; Uba1; |
| | Uba3; CHIP28 (Aqp1, Aquaporin 1); Uchl1; |
| | Uchl3; APP |
| Inflammation | IL-10; IL-1 (IL-1a; IL-1b); IL-13; IL-17 (IL-17a |
| | (CTLA8); IL-17b; IL-17c; IL-17d; IL-17f); |
| | II-23; Cx3cr1; ptpn22; TNFa; NOD2/CARD15 |
| | for IBD; IL-6; IL-12 (IL-12a; IL- |
| | 12b); CTLA4; Cx3cl1 |
| Parkinson's Disease | x-Synuclein; DJ-1; LRRK2; Parkin; PINK1 |

TABLE B
Diseases, Disorders and their associated genes

| DISEASE | |
|---|---|
| CATEGORY | DISEASE AND ASSOCIATED GENES |
| Blood and | Anemia (CDAN1, CDA1, RPS19, DBA, PKLR, PK1, |
| coagulation | NT5C3, UMPH1, PSN1, RHAG, RH50A, NRAMP2, |
| diseases and | SPTB, ALAS2, ANH1, ASB, ABCB7, ABC7, ASAT); |
| disorders | Bare lymphocyte syndrome (TAPBP, TPSN, TAP2, |
| | ABCB3, PSF2, RING11, MHC2TA, C2TA, RFX5, |
| | RFXAP, RFX5), Bleeding disorders (TBXA2R, P2RX1, |
| | P2X1); Factor H and factor H-like 1 (HF1, CFH, HUS); |
| | Factor V and factor VIII (MCFD2); Factor VII deficiency |
| | (F7); Factor X deficiency (F10); Factor XI deficiency |
| | (F11); Factor XII deficiency (F12, HAF); Factor XIIIA |
| | deficiency (F13A1, F13A); Factor XIIIB deficiency |
| | (F13B); Fanconi anemia (FANCA, FACA, FA1, FA, FAA, |
| | FAAP95, FAAP90, FLJ34064, FANCB, FANCC, FACC, |
| | BRCA2, FANCD1, FANCD2, FANCD, FACD, FAD, |
| | FANCE, FACE, FANCF, XRCC9, FANCG, BRIP1, |
| | BACH1, FANCJ, PHF9, FANCL, FANCM, KIAA1596); |
| | Hemophagocytic lymphohistiocytosis disorders (PRF1, |
| | HPLH2, UNC13D, MUNC13-4, HPLH3, HLH3, FHL3); |
| | Hemophilia A (F8, F8C, HEMA); Hemophilia B (F9, |
| | HEMB), Hemorrhagic disorders (PI, ATT, F5); Leukocyde |
| | deficiencies and disorders (ITGB2, CD18, LCAMB, LAD, |
| | EIF2B1, EIF2BA, EIF2B2, EIF2B3, EIF2B5, LVWM, |
| | CACH, CLE, EIF2B4); Sickle cell anemia (HBB); |
| | Thalassemia (HBA2, HBB, HBD, LCRB, HBA1) |
| Cell | B-cell non-Hodgkin lymphoma (BCL7A, BCL7); |
| dysregulation | Leukemia (TAL1, TCL5, SCL, TAL2, FLT3, NBS1, NBS, |
| and | ZNFN1A1, IK1, LYF1, HOXD4, HOX4B, BCR, CML, |
| oncology | PHL, ALL, ARNT, KRAS2, RASK2, GMPS, AF10, |
| diseases and | ARHGEF12, LARG, KIAA0382, CALM, CLTH, CEBPA, |
| disorders | CEBP, CHIC2, BTL, FLT3, KIT, PBT, LPP, NPM1, |
| | NUP214, D9S46E, CAN, CAIN, RUNX1, CBFA2, |
| | AML1, WHSCIL1, NSD3, FLT3, AF1Q, NPM1, NUMA1, |
| | ZNF145, PLZF, PML, MYL, STAT5B, AF10, CALM, |
| | CLTH, ARL11, ARLTS1, P2RX7, P2X7, BCR, CML, |
| | PHL, ALL, GRAF, NF1, VRNF, WSS, NFNS, PTPN11, |
| | PTP2C, SHP2, NS1, BCL2, CCND1, PRAD1, BCL1, |
| | TCRA, GATA1, GF1, ERYF1, NFE1, ABL1, NQO1, |
| | DIA4, NMOR1, NUP214, D9S46E, CAN, CAIN) |
| Inflammation | AIDS (KIR3DL1, NKAT3, NKB1, AMB11, KIR3DS1, |
| and immune | IFNG, CXCL12, SDF1); Autoimmune lymphoproliferative |
| related | syndrome (TNFRSF6, APT1, FAS, CD95, ALPS1A); |
| diseases | Combined immunodeficiency, (IL2RG, SCIDX1, SCIDX, |
| and | IMD4); HIV-1 (CCL5, SCYA5, D17S136E, TCP228), HIV |
| disorders | susceptibility or infection (IL10, CSIF, CMKBR2, CCR2, |
| | CMKBR5, CCCKR5 (CCR5)); Immunodeficiencies |
| | (CD3E, CD3G, AICDA, AID, HIGM2, TNFRSF5, CD40, |
| | UNG, DGU, HIGM4, TNFSF5, CD40LG, HIGM1, IGM, |
| | FOXP3, IPEX, AIID, XPID, PIDX, TNFRSF14B, TACI); |
| | Inflammation (IL-10, IL-1 (IL-1a, IL-1b), IL-13, IL-17 (IL- |
| | 17a (CTLA8), IL-17b, IL-17c, IL-17d, IL-17f), II-23, |
| | Cx3cr1, ptpn22, TNFa, NOD2/CARD15 for IBD, IL-6, IL- |
| | 12 (IL-12a, IL-12b), CTLA4, Cx3cl1); Severe combined |
| | immunodeficiencies (SCIDs)(JAK3, JAKL, DCLREIC, |
| | ARTEMIS, SCIDA, RAG1, RAG2, ADA, PTPRC, CD45, |
| | LCA, IL7R, CD3D, T3D, IL2RG, SCIDX1, SCIDX, |
| | IMD4) |
| Metabolic, | Amyloid neuropathy (TTR, PALB); Amyloidosis (APOA1, |
| liver, kidney | APP, AAA, CVAP, AD1, GSN, FGA, LYZ, TTR, PALB); |
| and protein | Cirrhosis (KRT18, KRT8, CIRHIA, NAIC, TEX292, |
| diseases and | KIAA1988); Cystic fibrosis (CFTR, ABCC7, CF, MRP7); |
| disorders | Glycogen storage diseases (SLC2A2, GLUT2, G6PC, |
| | G6PT, G6PT1, GAA, LAMP2, LAMPB, AGL, GDE, |
| | GBE1, GYS2, PYGL, PFKM); Hepatic adenoma, 142330 |
| | (TCF1, HNF1A, MODY3), Hepatic failure, early onset, |
| | and neurologic disorder (SCOD1, SCO1), Hepatic lipase |
| | deficiency (LIPC), Hepatoblastoma, cancer and carcinomas |
| | (CTNNB1, PDGFRL, PDGRL, PRLTS, AXIN1, AXIN, |
| | CTNNB1, TP53, P53, LFS1, IGF2R, MPRI, MET, CASP8, |
| | MCH5; Medullary cystic kidney disease (UMOD, HNFJ, |
| | FJHN, MCKD2, ADMCKD2); Phenylketonuria (PAH, |
| | PKU1, QDPR, DHPR, PTS); Polycystic kidney and hepatic |
| | disease (FCYT, PKHD1, ARPKD, PKD1, PKD2, PKD4, |
| | PKDTS, PRKCSH, G19P1, PCLD, SEC63) |
| Muscular/ | Becker muscular dystrophy (DMD, BMD, MYF6), |
| Skeletal | Duchenne Muscular Dystrophy (DMD, BMD); Emery- |
| diseases | Dreifuss muscular dystrophy (LMNA, LMN1, EMD2, |
| and | FPLD, CMD1A, HGPS, LGMD1B, LMNA, LMN1, |
| disorders | EMD2, FPLD, CMD1A); Facioscapulohumeral muscular |
| | dystrophy (FSHMD1A, FSHD1A); Muscular dystrophy |
| | (FKRP, MDC1C, LGMD2I, LAMA2, LAMM, LARGE, |
| | KIAA0609, MDC1D, FCMD, TTID, MYOT, CAPN3, |
| | CANP3, DYSF, LGMD2B, SGCG, LGMD2C, DMDA1, |
| | SCG3, SGCA, ADL, DAG2, LGMD2D, DMDA2, SGCB, |
| | LGMD2E, SGCD, SGD, LGMD2F, CMD1L, TCAP, |
| | LGMD2G, CMDIN, TRIM32, HT2A, LGMD2H, FKRP, |
| | MDC1C, LGMD2I, TTN, CMD1G, TMD, LGMD2J, |
| | POMT1, CAV3, LGMDIC, SEPN1, SELN, RSMD1, |
| | PLEC1, PLTN, EBS1); Osteopetrosis (LRP5, BMND1, |
| | LRP7, LR3, OPPG, VBCH2, CLCN7, CLC7, OPTA2, |
| | OSTMI, GL, TCIRG1, TIRC7, OC116, OPTB1); Muscular |
| | atrophy (VAPB, VAPC, ALS8, SMN1, SMA1, SMA2, |
| | SMA3, SMA4, BSCL2, SPG17, GARS, SMAD1, CMT2D, |
| | HEXB, IGHMBP2, SMUBP2, CATF1, SMARD1) |
| Dermato- | Albinisim (TYR, OCA2, TYRP1, SLC45A2, LYST), |
| logical | Ectodermal dysplasias (EDAR, EDARADD, WNT10A), |
| diseases | Ehlers-Danlos syndrome (COL5A1, COL5A2, COL1A1, |
| and | COL1A2, COL3A1, TNXB, ADAMTS2, PLOD1, |
| disorders | FKBP14), Ichthyosis-associated disorders (FLG, STS, |
| | TGM1, ALOXE3/ALOX12B, KRT1, KRT10, ABCA12, |
| | KRT2, GJB2, TGM1, ABCA12, CYP4F22, ALOXE3, |
| | CERS3, NSHDL, EBP, MBTPS2, GJB2, SPINK5, |
| | AGHD5, PHYH, PEX7, ALDH3A2, ERCC2, ERCC3, |
| | GFT2H5, GBA), Incontinentia pigmenti (IKBKG, NEMO), |
| | Tuberous sclerosis (TSC1, TSC2), Premature aging |
| | syndromes (POLR3A, PYCR1, LMNA, POLD1, WRN, |
| | DMPK) |
| Neurological | ALS (SOD1, ALS2, STEX, FUS, TARDBP, VEGF |
| and | (VEGF-a, VEGF-b, VEGF-c); Alzheimer disease (APP, |
| Neuronal | AAA, CVAP, AD1, APOE, AD2, PSEN2, AD4, STM2, |
| diseases and | APBB2, FE65L1, NOS3, PLAU, URK, ACE, DCP1, |
| disorders | ACE1, MPO, PACIP1, PAXIPIL, PTIP, A2M, BLMH, |
| | BMH, PSEN1, AD3); Autism (Mecp2, BZRAP1, MDGA2, |
| | Sema5A, Neurexin 1, GLO1, MECP2, RTT, PPMX, |
| | MRX16, MRX79, NLGN3, NLGN4, KIAA1260, |
| | AUTSX2); Fragile X Syndrome (FMR2, FXR1, FXR2, |
| | mGLUR5); Huntington's disease and disease like disorders |
| | (HD, IT15, PRNP, PRIP, JPH3, JP3, HDL2, TBP, SCA17); |
| | Parkinson disease (NR4A2, NURR1, NOT, TINUR, |
| | SNCAIP, TBP, SCA17, SNCA, NACP, PARK1, PARK4, |
| | DJ1, PARK7, LRRK2, PARK8, PINK1, PARK6, UCHL1, |
| | PARK5, SNCA, NACP, PARK1, PARK4, PRKN, PARK2, |
| | PDJ, DBH, NDUFV2); Rett syndrome (MECP2, RTT, |
| | PPMX, MRX16, MRX79, CDKL5, STK9, MECP2, RTT, |
| | PPMX, MRX16, |
| | MRX79, x-Synuclein, DJ-1); Schizophrenia (Neuregulin1 |
| | (Nrg1), Erb4 (receptor for Neuregulin), Complexin1 |
| | (Cplx1), Tph1 Tryptophan hydroxylase, Tph2, Tryptophan |
| | hydroxylase 2, Neurexin 1, GSK3, GSK3a, GSK3b, 5-HTT |
| | (Slc6a4), COMT, DRD (Drdla), SLC6A3, DAOA, |
| | DTNBP1, Dao (Dao1)); Secretase Related Disorders |
| | (APH-1 (alpha and beta), Presenilin (Psen1), nicastrin, |
| | (Ncstn), PEN-2, Nos1, Parp1, Natl, Nat2); Trinucleotide |
| | Repeat Disorders (HTT (Huntington's Dx), |
| | SBMA/SMAX1/AR (Kennedy's Dx), FXN/X25 |
| | (Friedrich's Ataxia), ATX3 (Machado-Joseph's Dx), |
| | ATXN1 and ATXN2 (spinocerebellar ataxias), DMPK |
| | (myotonic dystrophy), Atrophin-1 and Atn1 (DRPLA Dx), |
| | CBP (Creb-BP - global instability), VLDLR (Alzheimer's), |
| | Atxn7, Atxn10) |
| Ocular | Age-related macular degeneration (Abcr, Ccl2, Cc2, cp |
| diseases and | (ceruloplasmin), Timp3, cathepsinD, Vldlr, Ccr2); Cataract |
| disorders | (CRYAA, CRYA1, CRYBB2, CRYB2, PITX3, BFSP2, |
| | CP49, CP47, CRYAA, CRYA1, PAX6, AN2, MGDA, |
| | CRYBA1, CRYB1, CRYGC, CRYG3, CCL, LIM2, MP19, |
| | CRYGD, CRYG4, BFSP2, CP49, CP47, HSF4, CTM, |
| | HSF4, CTM, MIP, AQP0, CRYAB, CRYA2, CTPP2, |
| | CRYBB1, CRYGD, CRYG4, CRYBB2, CRYB2, CRYGC, |
| | CRYG3, CCL, CRYAA, CRYA1, GJA8, CX50, CAE1, |
| | GJA3, CX46, CZP3, CAE3, CCM1, CAM, KRIT1); |
| | Corneal clouding and dystrophy (APOA1, TGFBI, CSD2, |
| | CDGG1, CSD, BIGH3, CDG2, TACSTD2, TROP2, M1S1, |
| | VSX1, RINX, PPCD, PPD, KTCN, COL8A2, FECD, |
| | PPCD2, PIP5K3, CFD); Cornea plana congenital (KERA, |
| | CNA2); Glaucoma (MYOC, TIGR, GLCIA, JOAG, |
| | GPOA, OPTN, GLCIE, FIP2, HYPL, NRP, CYP1B1, |
| | GLC3A, OPA1, NTG, NPG, CYP1B1, GLC3A); Leber |
| | congenital |
| | amaurosis (CRB1, RP12, CRX, CORD2, CRD, RPGRIP1, |
| | LCA6, CORD9, RPE65, RP20, AIPL1, LCA4, GUCY2D, |
| | GUC2D, LCA1, CORD6, RDH12, LCA3); Macular |
| | dystrophy (ELOVL4, ADMD, STGD2, STGD3, RDS, |
| | RP7, PRPH2, PRPH, AVMD, AOFMD, VMD2) |

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR molecule, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

As used herein the term “wild-type” is a term of the art understood by skilled persons and means the typical form of an organism, strain, gene or characteristic as it occurs in nature as distinguished from mutant or variant forms. Accordingly, as used herein, where a sequence of amino acids or nucleotides refers to a wild-type sequence, a variant refers to variant of that sequence, e.g., comprising substitutions, deletions, insertions. In embodiments of the present invention, an engineered CRISPR nuclease is a variant CRISPR nuclease comprising at least one amino acid modification (e.g., substitution, deletion, and/or insertion), also referred to as a “mutation,” compared to the wild-type OMNI-79 nuclease of SEQ ID NO: 1.

The terms “non-naturally occurring” or “engineered” are used interchangeably and indicate human manipulation. The terms, when referring to nucleic acid molecules or polypeptides may mean that the nucleic acid molecule or the polypeptide is at least substantially free from at least one other component with which they are naturally associated in nature and as found in nature.

The terms “mutant” or “variant” are used interchangeably and indicate a molecule that is non-naturally occurring or engineered.

As used herein the term “amino acid” includes natural and/or unnatural or synthetic amino acids, including glycine and both the D- or L-, optical isomers, and amino acid analogs and peptidomimetics.

As used herein, “genomic DNA” refers to linear and/or chromosomal DNA and/or to plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of DNA sequences at the target site(s) in a genome.

“Eukaryotic” cells include, but are not limited to, fungal cells (such as yeast), plant cells, animal cells, mammalian cells and human cells.

As used herein, the term “modified cells” refers to cells in which a double strand break is affected by a complex of an RNA molecule and the CRISPR nuclease variant as a result of hybridization with the target sequence, i.e. on-target hybridization. The term “modified cells” may further encompass cells in which a repair or correction of a mutation was affected following the double strand break induced by the variant. The modified cell may be any type of cell e.g., eukaryotic or prokaryotic, in any environment e.g., isolated or not, maintained in culture, in vitro, ex vivo, in vivo or in planta.

This invention provides a modified cell or cells obtained by use of any of the variants or methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cell (HSC), or any cell suitable for an allogenic cell transplant or autologous cell transplant. The variants and methods described herein may also be utilized to generate chimeric antigen receptor T (CAR-T) cells.

This invention also provides a composition comprising these modified cells and a pharmaceutically acceptable carrier. Also provided is an in vitro or ex vivo method of preparing this, comprising mixing the cells with the pharmaceutically acceptable carrier.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity.

The terms “protospacer adjacent motif” or “PAM” as used herein refers to a nucleotide sequence of a target DNA located in proximity to the targeted DNA sequence and recognized by the CRISPR nuclease. The PAM sequence may differ depending on the nuclease identity. For example, wild-type Streptococcus pyogenes Cas9 recognizes a “NGG” PAM sequence. A skilled artisan will appreciate that single-guide RNA molecules or crRNA: tracrRNA complexes capable of complexing with a CRISPR nuclease such as to associate with a target genomic DNA sequence of interest next to a protospacer adjacent motif (PAM). The nuclease then mediates cleavage of target DNA to create a double-stranded break within the protospacer.

As used herein, a sequence or molecule has an X % “sequence identity” to another sequence or molecule if X % of bases or amino acids between the sequences of molecules are the same and in the same relative position. For example, a first nucleotide sequence having at least a 95% sequence identity with a second nucleotide sequence will have at least 95% of bases, in the same relative position, identical with the other sequence.

The terms “nuclear localization sequence” and “NLS” are used interchangeably to indicate an amino acid sequence/peptide that directs the transport of a protein with which it is associated from the cytoplasm of a cell across the nuclear envelope barrier. The term “NLS” is intended to encompass not only the nuclear localization sequence of a particular peptide, but also derivatives thereof that are capable of directing translocation of a cytoplasmic polypeptide across the nuclear envelope barrier. NLSs are capable of directing nuclear translocation of a polypeptide when attached to the N-terminus, the C-terminus, or both the N- and C-termini of the polypeptide. In addition, a polypeptide having an NLS coupled by its N- or C-terminus to amino acid side chains located randomly along the amino acid sequence of the polypeptide will be translocated. Typically, an NLS consists of one or more short sequences of positively charged lysines or arginines exposed on the protein surface, but other types of NLS are known. Non-limiting examples of NLSs include an NLS sequence derived from: the SV40 virus large T-antigen, nucleoplasmin, c-myc, the hRNPAI M9 NLS, the IBB domain from importin-alpha, myoma T protein, human p53, mouse c-abl IV, influenza vims NS1, Hepatitis virus delta antigen, mouse Mx1 protein, human poly (ADP-ribose) polymerase, and the steroid hormone receptors (human) glucocorticoid.

The term “CRISPR system” refers to a CRISPR endonuclease system that includes a CRISPR nuclease protein, such as the mutants or variants described herein, and a suitable guide RNA molecule or guide RNA complex, e.g. a single-guide RNA or a crRNA: tracrRNA complex, for targeting the CRISPR nuclease protein to a desired target DNA sequence based on complementarity between a portion of the guide RNA molecule or guide RNA complex and the target DNA sequence. The term “wild-type CRISPR endonuclease system” refers to a CRISPR endonuclease system that includes wild-type CRISPR protein and a suitable guide RNA molecule or guide RNA complex, e.g. a single-guide RNA or a crRNA: tracrRNA complex, for targeting the wild-type CRISPR nuclease protein to a desired target DNA sequence based on complementarity between a portion of the guide RNA molecule or guide RNA complex and the target DNA sequence.

In the context of the invention, “maintained on-target editing activity” refers to the ability of an OMNI-79 variant to target a DNA target site that is targeted by a guide RNA molecule associated with, and thereby programming, the OMNI-79 variant. In some embodiments, the OMNI-79 variant maintains on-target editing activity of a DNA target at a percent editing level greater than or equal to the percent editing level of a wild-type OMNI-79 nuclease for the DNA target. In some embodiments, the OMNI-79 variant maintains on-target editing activity of a DNA target of at least 100%, 95%, 90%, 80%, 70%, 60%, 50%, 40%, or 30% the level of percent editing of a wild-type OMNI-79 nuclease for the DNA target.

For the foregoing embodiments, each embodiment disclosed herein is contemplated as being applicable to each of the other disclosed embodiment. For example, it is understood that any of the RNA molecules or compositions of the present invention may be utilized in any of the methods of the present invention.

As used herein, all headings are simply for organization and are not intended to limit the disclosure in any manner. The content of any individual section may be equally applicable to all sections.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Generally, the nomenclature used herein, and the laboratory procedures utilized in the present invention include molecular, biochemical, microbiological and recombinant DNA techniques. Such techniques are thoroughly explained in the literature. See, for example, Sambrook et al., “Molecular Cloning: A laboratory Manual” (1989); Ausubel, R. M. (Ed.), “Current Protocols in Molecular Biology” Volumes I-III (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (Eds.), “Genome Analysis: A Laboratory Manual Series”, Vols. 1-4, Cold Spring Harbor Laboratory Press, New York (1998); Methodologies as set forth in U.S. Pat. Nos. 4,666,828; 4,683,202; 4,801,531; 5,192,659 and 5,272,057; Cellis, J. E. (Ed.), “Cell Biology: A Laboratory Handbook”, Volumes I-III (1994); Freshney, “Culture of Animal Cells-A Manual of Basic Technique” Third Edition, Wiley-Liss, N. Y. (1994); Coligan J. E. (Ed.), “Current Protocols in Immunology” Volumes I-III (1994); Stites et al. (Eds.), “Basic and Clinical Immunology” (8th Edition), Appleton & Lange, Norwalk, CT (1994); Mishell and Shiigi (Eds.), “Strategies for Protein Purification and Characterization-A Laboratory Course Manual” CSHL Press (1996); Clokie and Kropinski (Eds.), “Bacteriophage Methods and Protocols”, Volume 1: Isolation, Characterization, and Interactions (2009), all of which are incorporated by reference. Other general references are provided throughout this document.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Example 1

### OMNI-79 CRISPR Nuclease Variant Library

The open reading frame of the wild-type OMNI-79 CRIPSR nuclease was codon optimized for human cell line expression (SEQ ID NO: 1) and cloned into a dual expression plasmid (pShuttle) that enables both bacterial and mammalian expression using a T7 or a CMV promoter, respectively. A full gene library with combinatorial random mutations along the full length OMNI-79 open reading frame (ORF) was constructed using incorporated oligos with an NNK degenerate codon at each position in the OMNI-79 sequence, resulting in a library having an average of 2.6 amino acid substitutions per ORF.

### Bacterial-Based Positive Selection Systems

In order to isolate OMNI-79 variants with enhanced activity, a positive selection bacterial system was designed. In this system, a positive selection plasmid was electroporated into Escherichia coli strain BW25141 (1DE3) to create the positive selection bacterial strain. The positive selection plasmid contains a T7-expressed single-guide RNA (sgRNA) and an embedded on-target site. The sequence of the target site, which is a sequence located within Exon 5 of the human Serpina gene, and the spacer and scaffold sequences of the guide RNA molecule, are listed in Table 1. The positive selection plasmid also contains a chloramphenicol resistance cassette and expresses the E. coli toxin gene CcdB under the control of an araBAD promoter. Accordingly, upon electroporating the OMNI-79 full gene library, which contains the OMNI-79 variants, into the positive selection bacterial strain, only bacterial colonies that express an active OMNI-79 variant which cleaves the positive selection plasmid, thus neutralizing the toxin, are able to survive on selective plates containing arabinose.

Following a 10-minute post-electroporation recovery period in TB media, transformed bacteria are plated on selective TB plates containing carbenicillin and 15 mM arabinose and incubated overnight at 37° C. The next morning the surviving pool is collected, plasmids are isolated, and re-transformed into the positive selection bacterial strain for another round of selection. Seven rounds of positive selection were performed, and after the final round single bacterial colonies were randomly picked and fully sequenced.

Target cleavage sites for bacterial selections

| | |
|---|---|
| SERPINA 1 Target | CACGTGGACCAGGC |
| site sequence | GACCACCGTGAAGG |
| (PAM in bold) | (SEQ ID NO: 5) |
| OMNI-79 Guide | CACGUGGACCAGGC |
| RNA molecule | GACCACCGUGA |
| 25-nucleotide | (SEQ ID NO: 6) |
| spacer sequence | |
| OMNI-79 Guide | GUUGCCGCUGGAGAA |
| RNA molecule | AUCCAGUUGUUAACA |
| scaffold | AGCAGCUUGACUGCA |
| sequence | CCAAAUAAGGCGGGG |
| | GCUGCGGCCCUCGC |
| | (SEQ ID NO: 7) |

To test the activity of the OMNI-79 nuclease variants after bacterial selection, positively-selected clones were isolated and their pShuttle plasmids were individually transfected into HeLa cells together with an sgRNA targeting the endogenous Serpina gene (Table 2). After 72 hours, cells were harvested, lysed, and their genomic DNA content was used in a PCR reaction which amplified the putative genomic target site sequence. Amplicons were then subjected to next-generation sequencing (NGS) and the resulting sequences were used to calculate the percentage of editing events in the target site. Short insertions or deletions (indels) around the cut site are the typical outcome of repair of DNA ends following nuclease-induced DNA cleavage. Therefore, the calculation of % editing was deduced from the fraction of indel-containing sequences within each amplicon.

Selected OMNI-79 nuclease variants were further analyzed with an additional guide RNA molecule targeting a mouse SARMI target site in Neuro-2a mouse neuroblastoma (Mn2A) cells (see Table 2).

Summary of guide RNA molecule
and genomic target sites

| | Spacer | | | |
|---|---|---|---|---|
| | Sequence | | | Genomic |
| | in | | | Target |
| Guide | Guide | | Genomic | Sequence |
| RNA | RNA | | Target | (PAM in |
| Name | molecule | Organism | Location | bold) |
| gSerpina_ | CACGUGG | Homo | Chr14 | CACGTGGA |
| 12 | ACCAGGC | Sapiens | 1497636- | CCAGGCGA |
| | GACCACC | | 1497663 | CCACCGTG |
| | GUGA | | HG_38 | AAGG |
| | (SEQ ID | | | (SEQ ID |
| | NO: 8) | | | NO: 9) |
| gmSARM1_ | GUGUUGG | Mus | Chr11 | GTGTTGGC |
| 57 | CUACCAA | Musculus | 78490806- | TACCAACA |
| | CAAGGAG | | 78490830 | AGGAGGTG |
| | G | | | G |
| | (SEQ | | | (SEQ ID |
| | ID NO: | | | NO: 11) |
| | 10) | | | |

### Results

Two OMNI-79 CRISPR nuclease variants that were highly enriched after the bacterial selection were isolated and both showed an increased activity relative to the wild-type OMNI-79 CRISPR nuclease in HeLa cells as indicated in Table 3. OMNI-79 Variant 5570 also showed high activity when tested in Neuro-2a mouse neuroblastoma (mN2A) cells using an mSARMI target (see Table 3).

TABLE 3
% Editing of Wild-Type OMNI-79, V5570, and V5603 Nuclease Variants in cells

| | | | | | % Editing | |
|---|---|---|---|---|---|---|
| | Substitutions | | | | in HeLa | |
| | Relative to | Amino Acid at | | | cells | % Editing |
| Nuclease | Wild-type | Position No. | | | Serpina | in mN2A cells |
| Name | OMNI-79 | 14 | 1005 | 1050 | Exon-5 | mSARM1 |
| WT OMNI-79 | — | I | S | E | 40 | 20 |
| V5570 | I14L and | L | R | E | 80 | 60 |
| | S1005R | | | | | |
| V5603 | S1005K and | I | K | K | 80 | N/A |
| | E1050K | | | | | |

### Example 2

### Results

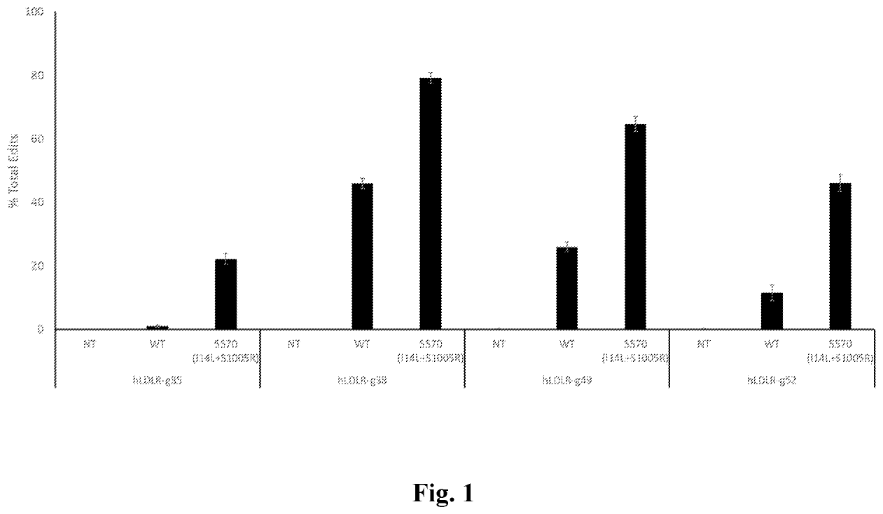

V5570 and V5603 are highly active variants of the CRISPR OMNI-79 nuclease (see Table 3, above). The editing activity of variant V5570 was tested on several targeted hLDLR gene sites using DNA transfection in HeLa cells (Table 6). In all cases OMNI-79 variant V5570 showed higher editing activity in comparison to wild-type OMNI-79 (FIG. 1, Table 8).

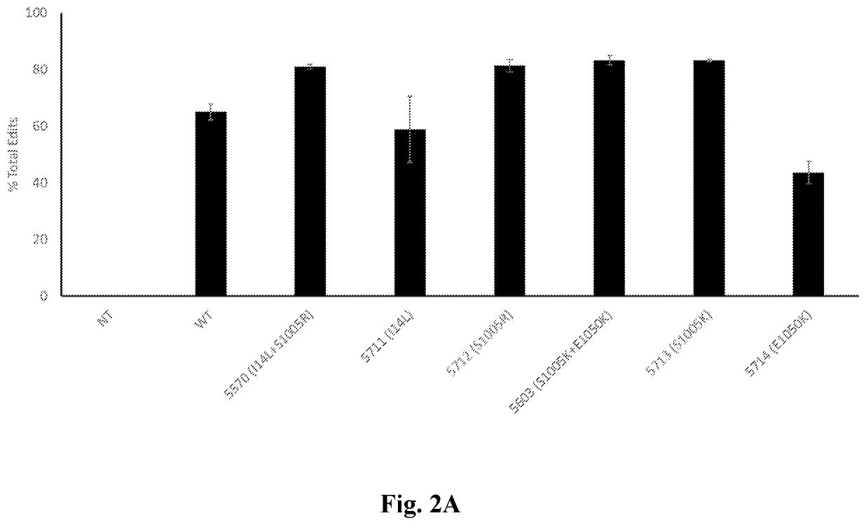

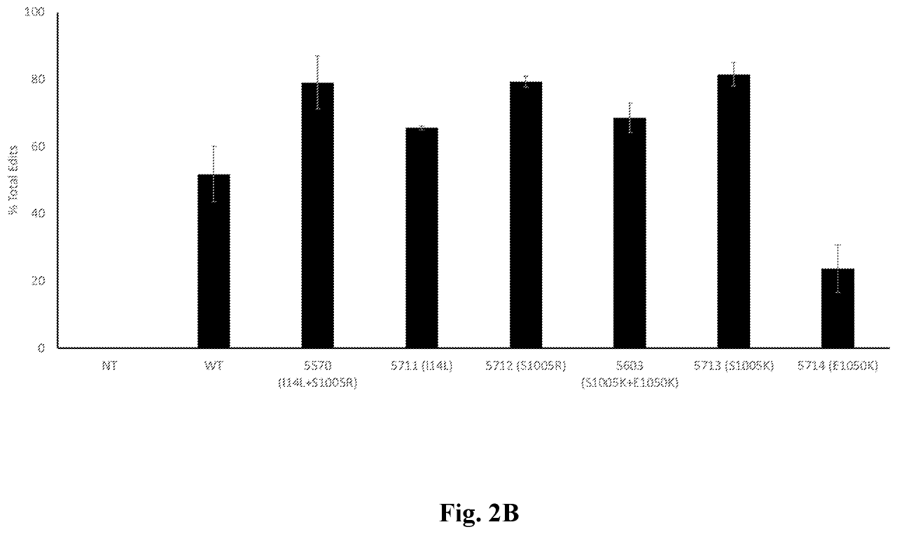

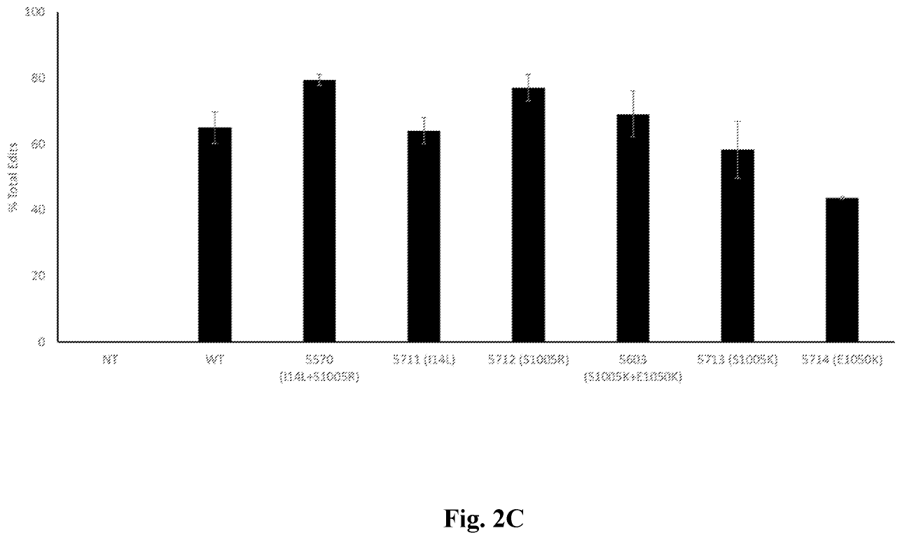

OMNI-79 variants V5570 and V5603 each contain two mutations. To investigate the effect and contribution to the activity of each mutation, single-mutation variants were generated and their activity was tested. Specifically, the activity of four single-mutation variants were tested via DNA transfection in HeLa cells on three targeted genomic sites: hSERP_g12R, hLDLR_g46 and hLDLR_g76 (Table 6). Only variants containing either a S1005R or S1005K mutation showed higher editing activity in comparison to wild-type OMNI-79 (FIGS. 2A-2C, Table 8). These results demonstrate that the mutations at position 1005 are responsible for the higher activity observed for variants V5570 and V5603.

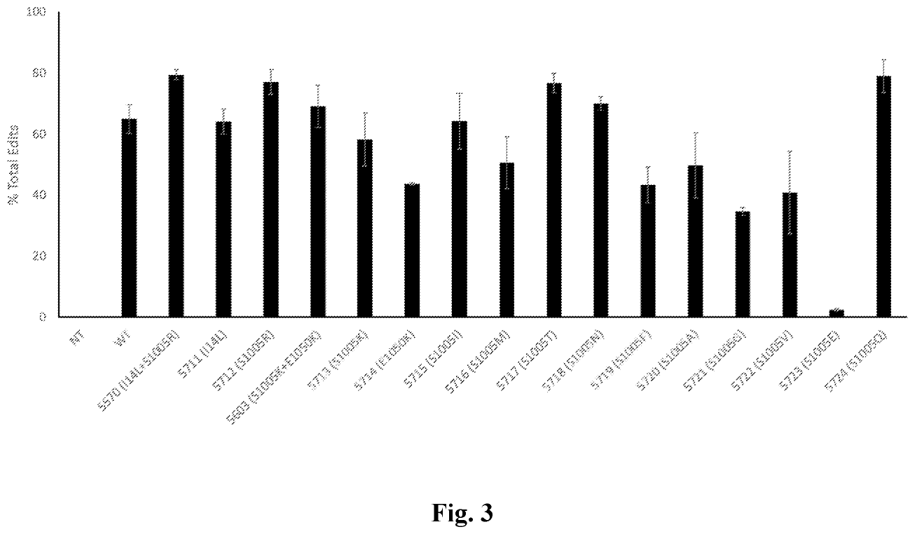

Once it was established that S1005R and S1005K are responsible for the observed activity of OMNI-79 V5570 and V5603, the effect of other substitutions at position 1005 were tested. An additional ten variants were generated, each containing a different amino acid substitution representing a different physicochemical group (Table 4). The effect of each single mutation on the variant's editing activity was tested as described above for the hLDLR_g76 target (FIG. 3, Table 6). The results demonstrated that positively charged amino acids, such as Arginine (R) and Lysine (K), at position 1005 all supported higher editing activity (FIG. 3, Table 8). In addition, polar amino acids such as Threonine (T), Asparagine (N), and Glutamine (Q) at position 1005 also supported higher editing activity (FIG. 3, Table 8).

### Materials and Methods

### DNA Transfection in Hela Cells

Nuclease activity in endogenous context in mammalian cells: OMNI-79 nuclease and its variant nucleases were expressed in a mammalian cell system (HeLa) by DNA transfection together with an sgRNA-expressing plasmid. Each sgRNA is composed of a tracrRNA portion and a spacer portion. The spacer 3′ genomic sequence contains the expected PAM relevant for OMNI-79 nuclease. All assays were performed in triplicate. ‘OMNI nuclease only’ (i.e. no guide) transfected cells served as a negative control. Cell lysates were used for site specific genomic DNA amplification and NGS analysis.

### NGS Analysis

At 72 h post-transfection, cells were harvested, and their genomic DNA content was used in a PCR reaction that amplified the corresponding putative genomic targets. Amplicons were subjected to NGS and the resulting sequences were then used to calculate the percentage of editing events in each target site. Short insertions or deletions (indels) around the cut site are the typical outcome of DNA repair following nuclease-induced DNA cleavage. The calculation of editing percentage was therefore deduced from the fraction of indel-containing sequences within each amplicon.

### Example 3

### Results

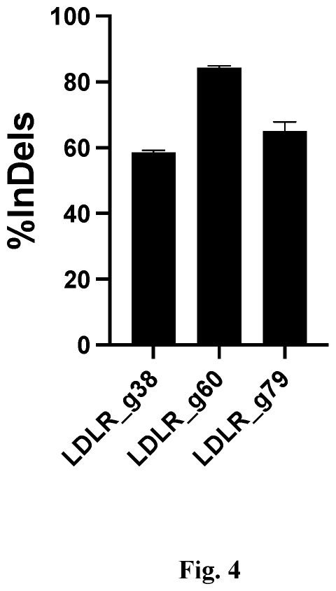

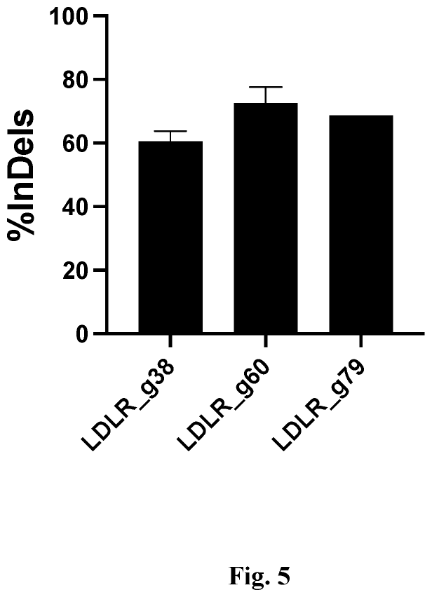

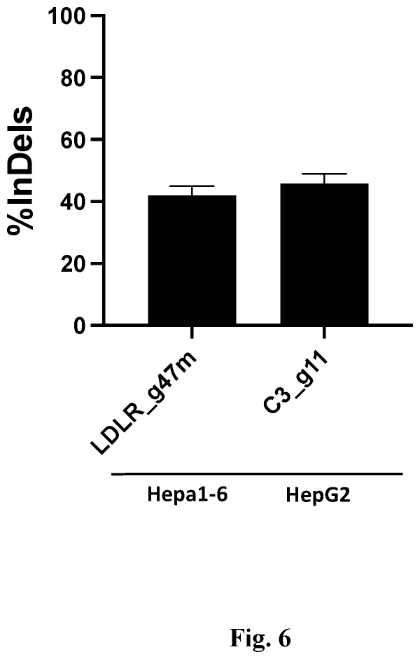

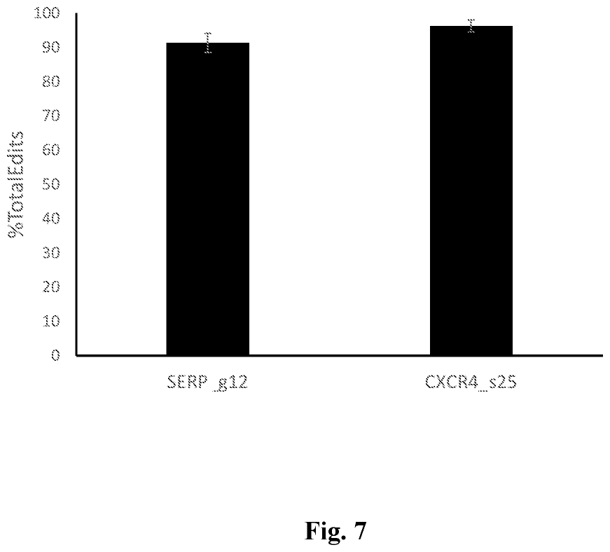

To test the potential of genomic editing by OMNI-79 V5570 several different delivery methods were employed. OMNI-79 V5570 was tested as part of a ribonucleoprotein (RNP) complex in HepG2 cells targeting several sites in the LDLR gene. In all cases the editing as calculated by NGS was high, ranging from 58.5% to up to 84.3% (FIG. 4, Table 6). Also, OMNI-79 V5570 was tested in transfection and electroporation experiments by delivering an mRNA molecule which encoded the variant nuclease. Again, in all cases a high level of editing was observed across three (3) different genes (FIG. 5, FIG. 7, Table 7). Finally, OMNI-79 V5570 was delivered using viral transduction (FIG. 6, Table 8).

### Materials and Methods

### Purification of OMNI-79 Protein

The expression method for protein production and synthetic guides production for the RNP assembly is as follows: Briefly, OMNI-79 nuclease open reading frame was codon optimized to bacteria (Table 1) and cloned into pNNC plasmid with the following elements-SV40 NLS-OMNI-79 ORF bacterial optimized—HA tag—SV40 NLS—8 His-tag (Table 4). The OMNI-79 construct was expressed in KRX cells (PROMEGA). Cells were grown in TB, 0.4% Glycerol with addition of 6.66 mM Rhamnose, 0.05% glucose and carb antibiotics. Induction was performed in mid-log phase, after 4 hours by temperature reduction to 18° C. Cells were lysed using chemical lysis and cleared lysate was purified on Ni-NTA resin. Next, purification using CEX (SO3 fractogel) resin followed by SEC purification on Superdex® 200 Increase 16/600, AKTA Pure (GE Healthcare Life Sciences), was performed. Fractions containing OMNI-79 protein in high salt concentration were pooled and concentrated to 22 mg/ml stocks and flash-frozen in liquid nitrogen and stored at −80° C.

### In Vitro Transcription (IVT) and Transfection

HiScribe T7 High Yield RNA Synthesis Kit (NEB #E2040S) was used with N1-Methylpseudouridine-5′-Triphosphate and CleanCap to produce OMNI-79 V5570-encoding mRNA, following the manufacture's instructions. The yield was approximately 150 μg mRNA. HeLa cells were seeded to be 70-90% confluent at the time of transfection. Lipofectamine 3000 was used to transfect the IVT mRNA with synthetic gRNAs, per the manufacturer's instructions. 72 hours after transfection, cells were dissociated with trypsin and genomic extract was prepared with QuickExtract solution according to manufacturer's instructions. InDels percentage analysis was performed by NGS.

RNP and mRNA Electroporation

For delivery of V5570 in RNP form, complexes were assembled by mixing 105 pmol of purified protein with 124 pmol of sgRNA and 100 μM Cas9 electroporation enhancer (IDT). After 10 minutes of incubation at 25° C., the RNP complexes were mixed with 4×10⁵ pre-washed HepG2 cells and electroporated using a Lonza SF Cell Line 4D-Nucleofector™ X Kit with the DS-123 program for HepG2 cells, according to the manufacturer's instructions. Cells were replated in 12-well tissue culture plates and incubated in a TC incubator (37° C., 5% CO₂). Cells were harvested 72 hours post-electroporation. Cell lysis and genomic DNA extraction was performed using Quick extract (Lucigen) and endogenous genomic regions were amplified using specific primers to measure on target activity by NGS (FIG. 4, Table 3).

For delivery of OMNI-79 V5570 in mRNA form, a solution of 1 μg of purified V5570 mRNA (Trilink) with 124 pmol of sgRNA and 100 μM Cas9 electroporation enhancer (IDT) was mixed. The solution was mixed with 4×10⁵ pre-washed HepG2 cells and electroporated using Lonza SF Cell Line 4D-Nucleofector™ X Kit with DS-123 program according to the manufacturer's instructions. Cells were replated in 12-well tissue culture plates, and incubated in TC incubator (37° C., 5% CO₂). Cells were harvested 72 hours post-electroporation. Cell lysis and genomic DNA extraction was performed in Quick extract (Lucigen) and endogenous genomic regions were amplified using specific primers to measure on target activity by NGS (FIG. 5, Table 3).

### Viral Transduction

For delivery of OMNI-79 V5570 in AAV form, AAV-DJ viruses that harbored OMNI-79 V5570 and corresponding sgRNA molecules were produced and purified (VectorBuilder). HepG2 and Hepa1-6 cells were infected by AAV-DJ harboring corresponding sgRNAs at an of MOI 1×10⁵ and 3×10⁵, respectively. Cells with the addition of viral particles were incubated in TC incubator (37° C., 5% CO₂) overnight. The next day cells were washed and incubated with fresh medium for another 48 hours. Cells were harvested 72 hours post-infection. Cell lysis and genomic DNA extraction was performed in Quick extract (Lucigen) and endogenous genomic regions were amplified using specific primers to measure on target activity by NGS (FIG. 6, Table 3).

TABLE 4
Amino acid substitutions of OMNI-79 nuclease variants

| | Substitutions | | | |
|---|---|---|---|---|
| | Relative to | Amino Acid at | | |
| Variant | Wild-type | Position No. | | |
| Name | OMNI-79 | 14 | 1005 | 1050 |
| WT OMNI-79 | — | I | S | E |
| V5711 | I14L | L | S | E |
| V5712 | S1005R | I | R | E |
| V5713 | S1005K | I | K | E |
| V5714 | E1050K | I | S | K |
| V5715 | S1005I | I | I | E |
| V5716 | S1005M | I | M | E |
| V5717 | S1005T | I | T | E |
| V5718 | S1005N | I | N | E |
| V5719 | S1005F | I | F | E |
| V5720 | S1005A | I | A | E |
| V5721 | S1005G | I | G | E |
| V5722 | S1005V | I | V | E |
| V5723 | S1005E | I | E | E |
| V5724 | S1005Q | I | Q | E |

TABLE 5
Tested sgRNA Sequences

| sgRNA | sgRNA Sequence |
|---|---|
| Name | (Spacer + Scaffold) |
| hSERPINA- | CACGUGGACCAGGuGACCACCGUGA |
| g12-Ref | GUUGCCGCUGGAGAAAUCCAGUUGU |
| | UAACAAGCAGCUUGACUGCACCAAA |
| | UAAGGCGGGGGCUGCGGCCCUCGCU |
| | UUUUU |
| | (SEQ ID NO: 40) |
| hLDLR-g46 | AGGAUAAGAGAAACAGGCCCGGGUU |
| | GCCGCUGGAGAAAUCCAGUUGUUAA |
| | CAAGCAGCUUGACUGCACCAAAUAA |
| | GGCGGGGGCUGCGGCCCUCGCUUUU |
| | UU |
| | (SEQ ID NO: 41) |
| hLDLR-g76 | GAAGCCACUCAUACAUACAACGGUU |
| | GCCGCUGGAGAAAUCCAGUUGUUAA |
| | CAAGCAGCUUGACUGCACCAAAUAA |
| | GGCGGGGGCUGCGGCCCUCGCUUUU |
| | UU |
| | (SEQ ID NO: 42) |
| hELANE-g35 | GCUGCAGUCCGGGCUGGGAGCGGGU |
| | GUUGCCGCUGGAGAAAUCCAGUUGU |
| | UAACAAGCAGCUUGACUGCACCAAA |
| | UAAGGCGGGGGCUGCGGCCCUCGCU |
| | UUUUU |
| | (SEQ ID NO: 43) |
| hLDLR-g35 | GGACAGUGCCCAUGCAAUGGCUGUU |
| | GCCGCUGGAGAAAUCCAGUUGUUAA |
| | CAAGCAGCUUGACUGCACCAAAUAA |
| | GGCGGGGGCUGCGGCCCUCGCUUUU |
| | UU |
| | (SEQ ID NO: 44) |
| hLDLR-g38 | AUCCCAACCCAAGCCAUUGCAUGUU |
| | GCCGCUGGAGAAAUCCAGUUGUUAA |
| | CAAGCAGCUUGACUGCACCAAAUAA |
| | GGCGGGGGCUGCGGCCCUCGCUUUU |
| | UU |
| | (SEQ ID NO: 45) |
| hLDLR-g49 | AAAACUUCCUGGAGAGAAAUGGGUU |
| | GCCGCUGGAGAAAUCCAGUUGUUAA |
| | CAAGCAGCUUGACUGCACCAAAUAA |
| | GGCGGGGGCUGCGGCCCUCGCUUUU |
| | UU |
| | (SEQ ID NO: 46) |
| hLDLR-g52 | AUGUUUGAGGAUUGUGUCACGGGUU |
| | GCCGCUGGAGAAAUCCAGUUGUUAA |
| | CAAGCAGCUUGACUGCACCAAAUAA |
| | GGCGGGGGCUGCGGCCCUCGCUUUU |
| | UU |
| | (SEQ ID NO: 47) |

TABLE 6
Target sites tested

| Target | DNA sequence | PAM (6 nt) |
|---|---|---|
| hSERPINA- | CACGTGGACC | AGGTGC |
| g12-Ref | AGGTGACCAC | |
| | CGTGA | |
| | (SEQ ID | |
| | NO: 48) | |
| hLDLR-g46 | AGGATAAGAG | GGGGAC |
| | AAACAGGCCC | |
| | GG | |
| | (SEQ ID | |
| | NO: 49) | |
| hLDLR-g76 | GAAGCCACTC | GGGACA |
| | ATACATACAA | |
| | CG | |
| | (SEQ ID | |
| | NO: 50) | |
| hELANE-g35 | GCTGCAGTCC | GGGGAC |
| | GGGCTGGGAG | |
| | CGGGT | |
| | (SEQ ID | |
| | NO: 51) | |
| hELANE- | AGTCCTGGCT | GGGGAG |
| g35-OT | GGGAGCAGGT | |
| | (SEQ ID | |
| | NO: 52) | |
| hLDLR-g35 | GGACAGTGCC | TGGGTT |
| | CATGCAATGG | |
| | CT | |
| | (SEQ ID | |
| | NO: 53) | |
| hLDLR-g38 | ATCCCAACCC | GGGCAC |
| | AAGCCATTGC | |
| | AT | |
| | (SEQ ID | |
| | NO: 54) | |
| hLDLR-g49 | AAAACTTCCT | AGGTGT |
| | GGAGAGAAAT | |
| | GG | |
| | (SEQ ID | |
| | NO: 55) | |
| hLDLR-g52 | ATGTTTGAGG | TGGAGA |
| | ATTGTGTCAC | |
| | GG | |
| | (SEQ ID | |
| | NO: 56) | |

TABLE 7
Plasmid details

| Plasmid | Purpose | Elements | Example |
|---|---|---|---|
| Shuttle | Expressing | T7 promoter-SV40 | Shuttle |
| V0 | OMNI | NLS-Linker- | V0:OMNI79_ |
| | polypeptide | OMNI ORF (Human | V5570 |
| | in the | optimized)- | (SEQ ID NO: 57) |
| | bacterial | HA Tag-SV40 | |
| | and | NLS-8XHisTag- | |
| | mammalian | T7 terminator | |
| | system | | |

TABLE 8
Activity in HeLa

| | | Sample | % Total | Standard |
|---|---|---|---|---|
| FIG. | Guide | Name | Edits | Deviation |
| FIG. 1 | hLDLR-g35 | NT | 0.01 | 0.00 |
| | | WT | 0.99 | 0.33 |
| | | 5570 | 22.17 | 1.68 |
| | hLDLR-g38 | NT | 0.01 | 0.00 |
| | | WT | 46.00 | 1.62 |
| | | 5570 | 79.17 | 1.61 |
| | hLDLR-g49 | NT | 0.25 | 0.00 |
| | | WT | 25.93 | 1.52 |
| | | 5570 | 64.73 | 2.37 |
| | hLDLR-g52 | NT | 0.25 | 0.00 |
| | | WT | 11.58 | 2.42 |
| | | 5570 | 46.12 | 2.71 |
| FIG. 2A | hSERPINA- | NT | 0.02 | 0.01 |
| | g12-Ref | WT | 65.05 | 2.84 |
| | | 5570 | 80.89 | 0.86 |
| | | 5711 | 58.85 | 11.64 |
| | | 5712 | 81.24 | 2.10 |
| | | 5603 | 83.22 | 1.68 |
| | | 5713 | 83.29 | 0.43 |
| | | 5714 | 43.70 | 4.02 |
| FIG. 2B | hLDLR-g46 | NT | 0.02 | 0.00 |
| | | WT | 51.81 | 8.21 |
| | | 5570 | 79.06 | 7.95 |
| | | 5711 | 65.57 | 0.60 |
| | | 5712 | 79.32 | 1.64 |
| | | 5603 | 68.50 | 4.39 |
| | | 5713 | 81.50 | 3.47 |
| | | 5714 | 23.66 | 7.11 |
| FIG. 2C | hLDLR-g76 | NT | 0.02 | 0.00 |
| | | WT | 64.96 | 4.71 |
| | | 5570 | 79.43 | 1.69 |
| | | 5711 | 64.07 | 4.04 |
| | | 5712 | 77.05 | 4.06 |
| | | 5603 | 69.08 | 6.90 |
| | | 5713 | 58.30 | 8.66 |
| | | 5714 | 43.71 | 0.31 |
| FIG. 3 | hLDLR-g76 | NT | 0.02 | 0.00 |
| | | WT | 64.96 | 4.71 |
| | | 5570 | 79.43 | 1.69 |
| | | 5711 | 64.07 | 4.04 |
| | | 5712 | 77.05 | 4.06 |
| | | 5603 | 69.08 | 6.90 |
| | | 5713 | 58.30 | 8.66 |
| | | 5714 | 43.71 | 0.31 |
| | | 5715 | 64.27 | 9.15 |
| | | 5716 | 50.63 | 8.49 |
| | | 5717 | 76.70 | 3.17 |
| | | 5718 | 70.03 | 2.17 |
| | | 5719 | 43.40 | 5.86 |
| | | 5720 | 49.69 | 10.62 |
| | | 5721 | 34.64 | 1.25 |
| | | 5722 | 40.82 | 13.62 |
| | | 5723 | 2.47 | 0.37 |
| | | 5724 | 78.92 | 5.38 |

TABLE 9
Sequences of OMNI-79 Variants

| | Amino Acid | |
|---|---|---|
| Nuclease Name | Sequence | DNA Sequence |
| Wild-type OMNI-79 | SEQ ID NO: 1 | SEQ ID NO: 4 |
| V5570 | SEQ ID NO: 2 | SEQ ID NO: 58 |
| V5603 | SEQ ID NO: 3 | SEQ ID NO: 59 |
| V5711 | SEQ ID NO: 12 | SEQ ID NO: 26 |
| V5712 | SEQ ID NO: 13 | SEQ ID NO: 27 |
| V5713 | SEQ ID NO: 14 | SEQ ID NO: 28 |
| V5714 | SEQ ID NO: 15 | SEQ ID NO: 29 |
| V5715 | SEQ ID NO: 16 | SEQ ID NO: 30 |
| V5716 | SEQ ID NO: 17 | SEQ ID NO: 31 |
| V5717 | SEQ ID NO: 18 | SEQ ID NO: 32 |
| V5718 | SEQ ID NO: 19 | SEQ ID NO: 33 |
| V5719 | SEQ ID NO: 20 | SEQ ID NO: 34 |
| V5720 | SEQ ID NO: 21 | SEQ ID NO: 35 |
| V5721 | SEQ ID NO: 22 | SEQ ID NO: 36 |
| V5722 | SEQ ID NO: 23 | SEQ ID NO: 37 |
| V5723 | SEQ ID NO: 24 | SEQ ID NO: 38 |
| V5724 | SEQ ID NO: 25 | SEQ ID NO: 39 |

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Atosuo J T, Lilius E-M. The Real-Time-Based Assessment of the Microbial Killing by the Antimicrobial Compounds of Neutrophils. Scientific World Journal. 2011; 11:2382-2390.
4. Basha et al. (2011) “Influence of Cationic Lipid Composition on Gene Silencing Properties of Lipid Nanoparticle Formulations of siRNA in Antigen-Presenting Cells”, Mol. Ther. 19 (12): 2186-200.
5. Behr (1994) “Gene transfer with synthetic cationic amphiphiles: Prospects for gene therapy”, Bioconjuage Chem 5:382-89.
6. Blaese et al. (1995) “Vectors in cancer therapy: how will they deliver”, Cancer Gene Ther. 2:291-97.
7. Blaese et al. (1995) “T lympocyte-directed gene therapy for ADA-SCID: initial trial results after 4 years”, Science 270 (5235): 475-80.
8. Briner et al. (2014) “Guide RNA functional modules direct Cas9 activity and orthognality”, Molecular Cell 56:333-39.
9. Buchschacher and Panganiban (1992) “Human immunodeficiency virus vectors for inducible expression of foreign genes”, J. Virol. 66:2731-39.
10. Burstein et al. (2017) “New CRISPR-Cas systems from uncultivated microbes”, Nature 542:237-41.
11. Canver et al., (2015) “BCL11A enhancer dissection by Cas9-mediated in situ saturating mutagenesis”, Nature Vol. 527, Pgs. 192-214.
12. Chang and Wilson (1987) “Modification of DNA ends can decrease end-joining relative to homologous recombination in mammalian cells”, Proc. Natl. Acad. Sci. USA 84:4959-4963.
13. Charlesworth et al. (2019) “Identification of preexisting adaptive immunity to Cas9 proteins in humans”, Nature Medicine, 25 (2), 249.
14. Chung et al. (2006) “Agrobacterium is not alone: gene transfer to plants by viruses and other bacteria”, Trends Plant Sci. 11 (1): 1-4.
15. Coelho et al. (2013) “Safety and efficacy of RNAi therapy for transthyretin amyloidosis” N. Engl. J. Med. 369, 819-829.
16. Crystal (1995) “Transfer of genes to humans: early lessons and obstacles to success”, Science 270 (5235): 404-10.
17. Dillon (1993) “Regulation gene expression in gene therapy” Trends in Biotechnology 11 (5): 167-173.
18. Dranoff et al. (1997) “A phase I study of vaccination with autologous, irradiated melanoma cells engineered to secrete human granulocyte macrophage colony stimulating factor”, Hum. Gene Ther. 8 (1): 111-23.
19. Dunbar et al. (1995) “Retrovirally marked CD34-enriched peripheral blood and bone marrow cells contribute to long-term engraftment after autologous transplantation”, Blood 85:3048-57.
20. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
21. Gao and Huang (1995) “Cationic liposome-mediated gene transfer” Gene Ther. 2 (10): 710-22.
22. Haddada et al. (1995) “Gene Therapy Using Adenovirus Vectors”, in: The Molecular Repertoire of Adenoviruses III: Biology and Pathogenesis, ed. Doerfler and Böhm, pp. 297-306.
23. Han et al. (1995) “Ligand-directed retro-viral targeting of human breast cancer cells”, Proc. Natl. Acad. Sci. USA 92 (21): 9747-51.
24. Humbert et al., (2019) “Therapeutically relevant engraftment of a CRISPR-Cas9-edited HSC-enriched population with HbF reactivation in nonhuman primates”, Sci. Trans. Med., Vol. 11, Pgs. 1-13.
25. Inaba et al. (1992) “Generation of large numbers of dendritic cells from mouse bone marrow cultures supplemented with granulocyte/macrophage colony-stimulating factor”, J Exp Med. 176 (6): 1693-702.
26. Jinek et al. (2012) “A programmable dual-RNA-guided DNA endonuclease in adaptive bacterial immunity”, Science 337 (6096): 816-21.
27. Johan et al. (1992) “GLVR1, a receptor for gibbon ape leukemia virus, is homologous to a phosphate permease of Neurospora crassa and is expressed at high levels in the brain and thymus”, J Virol 66 (3): 1635-40.
28. Judge et al. (2006) “Design of noninflammatory synthetic siRNA mediating potent gene silencing in vivo”, Mol Ther. 13 (3): 494-505.
29. Kohn et al. (1995) “Engraftment of gene-modified umbilical cord blood cells in neonates with adnosine deaminase deficiency”, Nature Medicine 1:1017-23.
30. Kremer and Perricaudet (1995) “Adenovirus and adeno-associated virus mediated gene transfer”, Br. Med. Bull. 51 (1): 31-44.
31. Macdiarmid et al. (2009) “Sequential treatment of drug-resistant tumors with targeted minicells containing siRNA or a cytotoxic drug”, Nat Biotehcnol. 27 (7): 643-51.
32. Makaryan V. et al. The diversity of mutations and clinical outcomes for ELANE-associated neutropenia. Curr. Opin. Hematol. 2015; 22 (1): 3-11.
33. Malech et al. (1997) “Prolonged production of NADPH oxidase-corrected granulocyes after gene therapy of chronic granulomatous disease”, PNAS 94 (22): 12133-38.
34. Maxwell et al. (2018) “A detailed cell-free transcription-translation-based assay to decipher CRISPR protospacer adjacent motifs”, Methods 14348-57
35. Miller et al. (1991) “Construction and properties of retrovirus packaging cells based on gibbon ape leukemia virus”, J Virol. 65 (5): 2220-24.
36. Miller (1992) “Human gene therapy comes of age”, Nature 357:455-60.
37. Mitani and Caskey (1993) “Delivering therapeutic genes-matching approach and application”, Trends in Biotechnology 11 (5): 162-66.
38. Nabel and Felgner (1993) “Direct gene transfer for immunotherapy and immunization”, Trends in Biotechnology 11 (5): 211-15.
39. Nasri M. et al. CRISPR/Cas9-mediated ELANE knockout enables neutrophilic maturation of primary hematopoietic stem and progenitor cells and induced pluripotent stem cells of severe congenital neutropenia patients. Haematologica. 2020; 105 (3): 598-609.
40. Nehls et al. (1996) “Two genetically separable steps in the differentiation of thymic epithelium” Science 272:886-889.
41. Remy et al. (1994) “Gene Transfer with a Series of Lipphilic DNA-Binding Molecules”, Bioconjugate Chem. 5 (6): 647-54.
42. Sentmanat et al. (2018) “A Survey of Validation Strategies for CRISPR-Cas9 Editing”, Scientific Reports 8:888, doi: 10.1038/s41598-018-19441-8.
43. Sommerfelt et al. (1990) “Localization of the receptor gene for type D simian retroviruses on human chromosome 19”, J. Virol. 64 (12): 6214-20.
44. Van Brunt (1988) “Molecular framing: transgenic animals as bioactors” Biotechnology 6:1149-54.
45. Vigne et al. (1995) “Third-generation adenovectors for gene therapy”, Restorative Neurology and Neuroscience 8 (1,2): 35-36.
46. Wagner et al. (2019) “High prevalence of Streptococcus pyogenes Cas9-reactive T cells within the adult human population” Nature Medicine, 25 (2), 242
47. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
48. Yu et al. (1994) “Progress towards gene therapy for HIV infection”, Gene Ther. 1 (1): 13-26.
49. Zetsche et al. (2015) “Cpf1 is a single RNA-guided endonuclease of a class 2 CRIPSR-Cas system” Cell 163 (3): 759-71.
50. Zuris et al. (2015) “Cationic lipid-mediated delivery of proteins enables efficient protein based genome editing in vitro and in vivo” Nat Biotechnol. 33 (1): 73-80.

What is claimed is:

1. A non-naturally occurring OMNI-79 nuclease variant having a wild-type OMNI-79 protein sequence (SEQ ID NO: 1) comprising an amino acid substitution in at least one of the following positions: S1005, I14, and E1050.

2. The OMNI-79 nuclease variant of claim 1, wherein the amino acid substitution at S1005 and/or E1050 are to an amino acid having a positively charged R-group.

3. The OMNI-79 nuclease variant of claim 2, wherein the amino acid having a positively charged R-group is lysine or arginine.

4. The OMNI-79 nuclease variant of claim 1, wherein the amino acid substitution is any one of the following substitutions: S1005R, S1005K, I14L, and E1050K.

5. The OMNI-79 nuclease variant of any one of claim 1 or 2, comprising an amino acid substitution at each of positions I14 and S1005.

6. The OMNI-79 nuclease variant of claim 3, wherein the amino acid substitutions are I14L and S1005R.

7. The OMNI-79 nuclease variant of any one of claim 1 or 2, comprising an amino acid substitution at each of positions S1005 and E1050.

8. The OMNI-79 nuclease variant claim 5, wherein the amino acid substitutions are S1005K and E1050K.

9. The OMNI-79 nuclease variant of claim 1, having an amino acid sequence of any one of SEQ ID NO: 2, SEQ ID NO: 3, or SEQ ID NOs: 12-25.

10. The OMNI-79 nuclease variant of claim 1, wherein the amino acid substitution is at I14 and is any one of the following substitutions: I14L, I14V, I14F, I14C, I14A, or I14T.

11. The OMNI-79 nuclease variant of claim 1, wherein the amino acid substitution is I14L.

12. The OMNI-79 nuclease variant of claim 1, wherein the amino acid substitution is at I14 and the amino acid has an aromatic or hydrophobic R-group.

13. The OMNI-79 nuclease variant of claim 1, wherein the amino acid substitution is at S1005 and is any one of the following substitutions: S1005R, S1005K, S1005Q, S1005I, S1005M, S1005V, S1005T, S1005N, S1005F, S1005A, S1005G, or S1005E.

14. The OMNI-79 nuclease variant of claim 1, wherein the amino acid substitution at S1005 is to an amino acid having a positively charged R-group.

15. The OMNI-79 nuclease variant of claim 1, wherein the amino acid substitution at S1005 is to an amino acid having a polar R-group.

16. The OMNI-79 nuclease variant of claim 1, wherein the amino acid substitution is S1005R.

17. The OMNI-79 nuclease variant of claim 1, wherein the amino acid substitution is S1005K.

18. The OMNI-79 nuclease variant of claim 1, wherein the amino acid substitution is S1005T.

19. The OMNI-79 nuclease variant of claim 1, wherein the amino acid substitution is S1005N.

20. The OMNI-79 nuclease variant of claim 1, wherein the amino acid substitution is S1005Q.

21. The OMNI-79 nuclease variant of claim 1, wherein the amino acid substitution is at E1050 and is any one of the following substitutions: E1050K, E1050R, E1050P, E1050A, E1050I, E1050L, E1050V, E1050G, or E1050T.

22. The OMNI-79 nuclease variant of claim 1, wherein the amino acid substitution is E1050K.

23. The OMNI-79 nuclease variant of claim 1, wherein the amino acid substitution at E1050 is to an amino acid having a positively charged R-group.

24. The OMNI-79 nuclease variant of any one of claims 1-23, having at least 80% sequence identity to the wild-type OMNI-79 protein sequence (SEQ ID NO:1).

25. The OMNI-79 nuclease variant of any one of claims 1-24, further comprising a nuclear localization sequence (NLS).

26. The OMNI-79 nuclease variant of any one of claims 1-25, wherein the variant exhibits increased activity at a DNA target site when complexed with a guide RNA molecule that targets the variant to the said DNA target site relative to a wild-type OMNI-79 nuclease complexed with the guide RNA molecule.

27. A CRISPR system comprising the OMNI-79 nuclease variant of any one of claims 1-26 complexed with a guide RNA molecule that targets a DNA target site, wherein the CRISPR system displays increased on-target editing activity relative to a wild-type CRISPR system comprising a wild-type OMNI-79 nuclease protein and the guide RNA molecule.

28. A method for gene editing having increased on-target editing activity, comprising contacting a DNA target site with an active CRISPR system comprising an OMNI-79 nuclease variant protein of any one of claims 1-26.

29. The method of claim 28, wherein the gene editing occurs in a eukaryotic cell or prokaryotic cell.

30. The method of claim 29, wherein the eukaryotic cell is a plant cell or mammalian cell.

31. The method of claim 30, wherein the mammalian cell is a human cell.

32. The method of any one of claims 28-31, wherein the DNA target site is located within or in proximity to a pathogenic allele of a gene.

33. The method of any one of claims 28-32, wherein the DNA target is repaired with an exogenous donor molecule.

34. The method of any one of claims 28-33, wherein the on-target editing activity is increased by at least 10%, 20%, 30%, 40%, 50%, 60%, 70%, 80%, 90%, 2-fold, 10-fold, 10²-fold, 10³-fold, 10⁴-fold, 10⁵-fold, or 10⁶-fold.

35. A modified cell obtained by the method of any one of claims 28-34.

36. The modified cell of claim 35, wherein the cell is capable of engraftment.

37. The modified cell of any one of claim 36 or 36, wherein the cell is capable of giving rise to progeny cells after engraftment.

38. The modified cell of any one of claims 35-37, wherein the cell is capable of giving rise to progeny cells after an autologous engraftment.

39. The modified cell of any one of claims 35-38, wherein the cell is capable of giving rise to progeny cells for at least 12 months or at least 24 months after engraftment.

40. The modified cell of any one of claims 35-39, wherein the cell is selected from the group consisting of a hematopoietic stem cell, a progenitor cell, a CD34+ hematopoietic stem cell, a bone marrow cell, and a peripheral mononucleated cell.

41. A composition comprising a modified cell of any one of claims 35-39 and a pharmaceutically acceptable carrier.

42. An in vitro or ex vivo method of preparing the composition of claim 41, comprising mixing the cells with the pharmaceutically acceptable carrier.

43. A polynucleotide molecule encoding the OMNI-79 variant nuclease of any one of claims 1-42.


SEQUENCE LISTING (59 sequences)

SEQ ID NO: 1 - protein, 1062 aa, Novosphingobium sp.
  source 1..1062: note = SYSU G00007
MEKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGSITFAPHNEAGPLKTRDTDPADPFRYVTTVASGLKKMRARQVRIDELGKVHDPGPRED

SEQ ID NO: 2 - protein, 1062 aa, synthetic construct
  REGION 1..1062: note = V5570 OMNI-79 Variant
MEKRLGLDIGTNSLGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGRITFAPHNEAGPLKTRDTDPADPFRYVTTVASGLKKMRARQVRIDELGKVHDPGPRED

SEQ ID NO: 3 - protein, 1062 aa, synthetic construct
  REGION 1..1062: note = V5603 OMNI-79 Variant
MEKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGKITFAPHNEAGPLKTRDTDPADPFRYVTTVASGLKKMRARQVRIDKLGKVHDPGPRED

SEQ ID NO: 4 - DNA, 3186 bp, Novosphingobium sp.
  source 1..3186: mol_type = other DNA; strain = SYSU G00007
atggagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcagcatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 5 - DNA, 28 bp, Homo sapiens
  source 1..28: mol_type = genomic DNA
cacgtggaccaggcgaccaccgtgaagg

SEQ ID NO: 6 - RNA, 25 nt, synthetic construct
  misc_feature 1..25: note = OMNI-79 Guide RNA molecule 25-nucleotide spacer sequence
  source 1..25: mol_type = other RNA
cacgtggaccaggcgaccaccgtga

SEQ ID NO: 7 - RNA, 74 nt, synthetic construct
  misc_feature 1..74: note = OMNI-79 Guide RNA molecule scaffold sequence
  source 1..74: mol_type = other RNA
gttgccgctggagaaatccagttgttaacaagcagcttgactgcaccaaataaggcgggggctgcggccctcgc

SEQ ID NO: 8 - RNA, 25 nt, synthetic construct
  misc_feature 1..25: note = gSerpina_12 Spacer Sequence in Guide RNA molecule
  source 1..25: mol_type = other RNA
cacgtggaccaggcgaccaccgtga

SEQ ID NO: 9 - DNA, 28 bp, Homo sapiens
  source 1..28: mol_type = genomic DNA
cacgtggaccaggcgaccaccgtgaagg

SEQ ID NO: 10 - RNA, 22 nt, synthetic construct
  misc_feature 1..22: note = gmSARM1_57 Spacer Sequence in Guide RNA molecule
  source 1..22: mol_type = other RNA
gtgttggctaccaacaaggagg

SEQ ID NO: 11 - DNA, 25 bp, Mus musculus
  source 1..25: mol_type = genomic DNA
gtgttggctaccaacaaggaggtgg

SEQ ID NO: 12 - protein, 1061 aa, synthetic construct
EKRLGLDIGTNSLGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGSITFAPHNEAGPLKTRDTDPADPFRYVTTVASGLKKMRARQVRIDELGKVHDPGPRED

SEQ ID NO: 13 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGRITFAPHNEAGPLKTRDTDPADPFRYVTTVASGLKKMRARQVRIDELGKVHDPGPRED

SEQ ID NO: 14 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGKITFAPHNEAGPLKTRDTDPADPFRYVTTVASGLKKMRARQVRIDELGKVHDPGPRED

SEQ ID NO: 15 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGSITFAPHNEAGPLKTRDTDPADPFRYVTTVASGLKKMRARQVRIDKLGKVHDPGPRED

SEQ ID NO: 16 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGIITFAPHNEAGPLKTRDTDPADPFRYVTTVASGLKKMRARQVRIDELGKVHDPGPRED

SEQ ID NO: 17 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGMITFAPHNEAGPLKTRDTDPADPFRYVTTVASGLKKMRARQVRIDELGKVHDPGPRED

SEQ ID NO: 18 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGTITFAPHNEAGPLKTRDTDPADPFRYVTTVASGLKKMRARQVRIDELGKVHDPGPRED

SEQ ID NO: 19 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGNITFAPHNEAGPLKTRDTDPADPFRYVTTVASGLKKMRARQVRIDELGKVHDPGPRED

SEQ ID NO: 20 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGFITFAPHNEAGPLKTRDTDPADPFRYVTTVASGLKKMRARQVRIDELGKVHDPGPRED

SEQ ID NO: 21 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGAITFAPHNEAGPLKTRDTDPADPFRYVTTVASGLKKMRARQVRIDELGKVHDPGPRED

SEQ ID NO: 22 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGGITFAPHNEAGPLKTRDTDPADPFRYVTTVASGLKKMRARQVRIDELGKVHDPGPRED

SEQ ID NO: 23 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGVITFAPHNEAGPLKTRDTDPADPFRYVTTVASGLKKMRARQVRIDELGKVHDPGPRED

SEQ ID NO: 24 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGEITFAPHNEAGPLKTRDTDPADPFRYVTTVASGLKKMRARQVRIDELGKVHDPGPRED

SEQ ID NO: 25 - protein, 1061 aa, synthetic construct
EKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGQITFAPHNEAGPLKTRDTDPADPFRYVTTVASGLKKMRARQVRIDELGKVHDPGPRED

SEQ ID NO: 26 - DNA, 3183 bp, synthetic construct
  source 1..3183: mol_type = other DNA
gagaagagactgggcctcgacatcggcacaaatagcttgggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcagcatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 27 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggccggatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 28 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcaagatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 29 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcagcatcacattcgccccacataatgaggccggacctctgaaaacccgggacaccgatcctgccgatcctttcagatacgtgacaacagtggccagcggcctgaagaagatgagagccagacaagtgcggatcgataagctgggcaaagtgcacgatcccggacctagagaggat

SEQ ID NO: 30 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcatcatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 31 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcatgatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 32 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcaccatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 33 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcaacatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 34 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcttcatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 35 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggagccatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 36 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggaggaatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 37 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggagtgatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 38 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggagagatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 39 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggccagatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 40 - RNA, 105 nt, synthetic construct
  source 1..105: mol_type = other RNA
cacgtggaccaggtgaccaccgtgagttgccgctggagaaatccagttgttaacaagcagcttgactgcaccaaataaggcgggggctgcggccctcgctttttt

SEQ ID NO: 41 - RNA, 102 nt, synthetic construct
  source 1..102: mol_type = other RNA
aggataagagaaacaggcccgggttgccgctggagaaatccagttgttaacaagcagcttgactgcaccaaataaggcgggggctgcggccctcgctttttt

SEQ ID NO: 42 - RNA, 102 nt, synthetic construct
  source 1..102: mol_type = other RNA
gaagccactcatacatacaacggttgccgctggagaaatccagttgttaacaagcagcttgactgcaccaaataaggcgggggctgcggccctcgctttttt

SEQ ID NO: 43 - RNA, 105 nt, synthetic construct
  source 1..105: mol_type = other RNA
gctgcagtccgggctgggagcgggtgttgccgctggagaaatccagttgttaacaagcagcttgactgcaccaaataaggcgggggctgcggccctcgctttttt

SEQ ID NO: 44 - RNA, 102 nt, synthetic construct
  source 1..102: mol_type = other RNA
ggacagtgcccatgcaatggctgttgccgctggagaaatccagttgttaacaagcagcttgactgcaccaaataaggcgggggctgcggccctcgctttttt

SEQ ID NO: 45 - RNA, 102 nt, synthetic construct
  source 1..102: mol_type = other RNA
atcccaacccaagccattgcatgttgccgctggagaaatccagttgttaacaagcagcttgactgcaccaaataaggcgggggctgcggccctcgctttttt

SEQ ID NO: 46 - RNA, 102 nt, synthetic construct
  source 1..102: mol_type = other RNA
aaaacttcctggagagaaatgggttgccgctggagaaatccagttgttaacaagcagcttgactgcaccaaataaggcgggggctgcggccctcgctttttt

SEQ ID NO: 47 - RNA, 102 nt, synthetic construct
  source 1..102: mol_type = other RNA
atgtttgaggattgtgtcacgggttgccgctggagaaatccagttgttaacaagcagcttgactgcaccaaataaggcgggggctgcggccctcgctttttt

SEQ ID NO: 48 - DNA, 25 bp, Homo sapiens
  source 1..25: mol_type = genomic DNA
cacgtggaccaggtgaccaccgtga

SEQ ID NO: 49 - DNA, 22 bp, Homo sapiens
  source 1..22: mol_type = genomic DNA
aggataagagaaacaggcccgg

SEQ ID NO: 50 - DNA, 22 bp, Homo sapiens
  source 1..22: mol_type = genomic DNA
gaagccactcatacatacaacg

SEQ ID NO: 51 - DNA, 25 bp, Homo sapiens
  source 1..25: mol_type = genomic DNA
gctgcagtccgggctgggagcgggt

SEQ ID NO: 52 - DNA, 20 bp, Homo sapiens
  source 1..20: mol_type = genomic DNA
agtcctggctgggagcaggt

SEQ ID NO: 53 - DNA, 22 bp, Homo sapiens
  source 1..22: mol_type = genomic DNA
ggacagtgcccatgcaatggct

SEQ ID NO: 54 - DNA, 22 bp, Homo sapiens
  source 1..22: mol_type = genomic DNA
atcccaacccaagccattgcat

SEQ ID NO: 55 - DNA, 22 bp, Homo sapiens
  source 1..22: mol_type = genomic DNA
aaaacttcctggagagaaatgg

SEQ ID NO: 56 - DNA, 22 bp, Homo sapiens
  source 1..22: mol_type = genomic DNA
atgtttgaggattgtgtcacgg

SEQ ID NO: 57 - DNA, 7507 bp, synthetic construct
  source 1..7507: mol_type = other DNA
taatacgactcactataggggaattgtgagcggataacaattcccaacaacaacaacaataaggaggtttttatggccatgcctaagaagaagagaaaggtgggtaccgagaagagactgggcctcgacatcggcacaaatagcttgggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggccggatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggatggatcctacccatacgatgttccagattacgcggccgctccaaaaaagaaaagaaaagttgcggctagccatcatcaccatcaccatcaccattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttacccggccgcataatgcttaagtcgaacagaaagtaatcgtattgtacacggccgcataatcacgtcgcgccgcttagcaataactagcataaccccttggggcctctaaacgggtcttgaggggttttttgacctaggctaggggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcctcgagtcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgacacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtcggggaattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaacaacaacaa

SEQ ID NO: 58 - DNA, 3183 bp, synthetic construct
  source 1..3183: mol_type = other DNA
gagaagagactgggcctcgacatcggcacaaatagcttgggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggccggatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 59 - DNA, 3183 bp, synthetic construct
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcaagatcacattcgccccacataatgaggccggacctctgaaaacccgggacaccgatcctgccgatcctttcagatacgtgacaacagtggccagcggcctgaagaagatgagagccagacaagtgcggatcgataagctgggcaaagtgcacgatcccggacctagagaggat